SEQUENCE LISTING (7 sequences)

SEQ ID NO: 1 - protein, 9 aa, Artificial Sequence
  Mutated Peptide
AMFWSVPTV

SEQ ID NO: 2 - protein, 9 aa, Artificial Sequence
  Mutated Peptide
CLNEYHLFL

SEQ ID NO: 3 - protein, 9 aa, Artificial Sequence
  Mutated Peptide
KMIGNHLWV

SEQ ID NO: 4 - protein, 9 aa, Artificial Sequence
  Mutated Peptide
FLYNLLTRV

SEQ ID NO: 5 - protein, 9 aa, Artificial Sequence
  Mutated Peptide
KLMNIQQKL

SEQ ID NO: 6 - protein, 9 aa, Artificial Sequence
  Mutated Peptide
IILVAVPHV

SEQ ID NO: 7 - protein, 9 aa, Artificial Sequence
  Mutated Peptide
MLGEQLFPL

(19) United States
(12) Patent Application Publication
(10) Pub. No.: US 2022/0305025 A1
(43) Pub. Date: Sep. 29, 2022

(54) PHOSPHATIDYLSERINE BINDING MOLECULES BLOCK IMMUNE SUPPRESSION OF TUMOR ASSOCIATED EXOSOMES

(71) Applicants: The Research Foundation for The State University of New York, Amherst, NY (US); Molecular Targeting Technologies, Inc., West Chester, PA (US); Immune Modulatory Therapies LLC, Eden, NY (US)

(72) Inventors: Richard B. BANKERT, Eden, NY (US); Koon Yan PAK, Malvern, PA (US); Brian D. GRAY, Exton, PA (US); Sathy V. BALU-IYER, Amherst, NY (US); Raymond KELLEHER, Amherst, NY (US); Gautam SHENOY, Amherst, NY (US); Maulasri BHATTA, Buffalo, NY (US)

(21) Appl. No.: 17/635,673

(22) PCT Filed: Aug. 17, 2020
(86) PCT No.: PCT/US20/46712

§ 371 Date: Feb. 15, 2022

Related U.S. Application Data
(60) Provisional application No. 62/887,588, filed on Aug. 15, 2019.

(51) Int. Cl. A61K 31/555; A61K 39/395; A61K 9/127; C07F 3/06; A61P 35/00

(52) U.S. Cl. CPC A61K 31/555; A61K 39/3955; A61K 9/127; C07F 3/06; A61P 35/00

(57) ABSTRACT

The present disclosure provides compounds that bind phosphatidylserine (PS). Also provided are compositions comprising the compounds and methods of using the compounds and/or compositions. The compounds and compositions may be used to treat an individual having or suspected of having cancer(s), infectious disease(s), chronic inflammation, and/or autoimmune condition(s).

### CROSS REFERENCE TO RELATED APPLICATIONS

This application claims priority to U.S. Provisional Application No. 62/887,588, filed on Aug. 15, 2019, the disclosure of which is incorporated herein by reference.

### STATEMENT REGARDING FEDERALLY SPONSORED RESEARCH

This invention was made with government support under contract no. CA131407 awarded by the National Institutes of Health. The government has certain rights in the invention.

### BACKGROUND OF THE DISCLOSURE

Previous studies have established that tumor-associated immune suppressive exosomes that are present in many different tumors are able to significantly arrest T cell function (Keller et al., Cancer Immunol. Res., 2015, 3(11): 1269-78). Most recently, it was reported that exosomes released from melanoma tumors in cancer patients suppress the function of CD8 T cells and facilitate tumor growth (Chen et al., Nature, 2018, 560(7718): 73-81). The exosomes are known to exhibit phosphatidylserine (PS) and the ganglioside GD3 on their surface. Previous attempts to block PS in cancer and infectious diseases in preclinical studies using anti-PS antibodies and annexin V, or to treat lung cancer in clinical trials using a PS specific antibody (bavituximab) (Birge et al., Cell Death Differ., 2016, 23(6): 962-78) have met with limited success owing to relatively low affinity PS-binding of the molecules used. Therefore, there is a need to develop drugs that will effective block the exosomal suppression of T cells.

### SUMMARY OF THE DISCLOSURE

The present disclosure provides compounds that bind phosphatidylserine (PS). Also provided are compositions comprising the compounds and methods of using the compounds and/or compositions.

In an aspect, the present disclosure provides compounds comprising a branching group having the following structure:

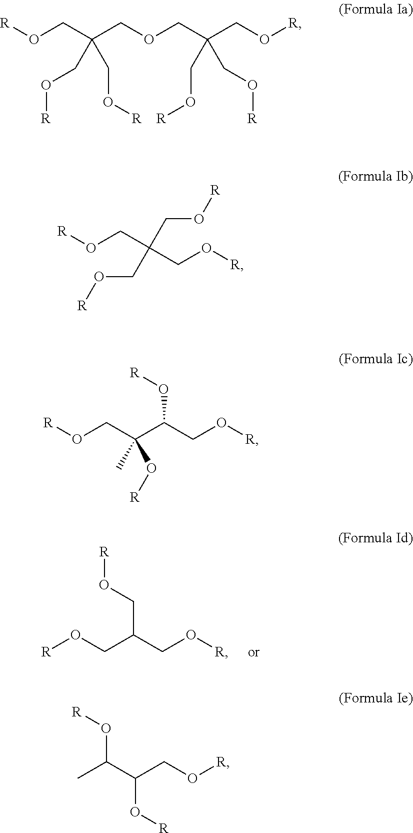

where each R is independently at each occurrence hydrogen or comprises a poly(ethylene glycol) (PEG) group or an ethylene glycol group, a linker group, and an end group. The compounds may also have various counter anions. One or more of the R groups may be the same or different. In various examples, one or more of the R groups are hydrogen (e.g., for Formula Ia: one, two, three, four, or five R groups may be hydrogen; for Formula Ib and Ic: one, two, or three R groups may be hydrogen, for Formulas Id and Ie: one or two R groups may be hydrogen).

An end group comprises various aryl groups, heteroaryl groups, a tertiary amine, and a plurality of divalent cations. The heteroaryl groups may have various substituents, such as, for example, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof. One, some, or all of the heteroaryl groups may be, for example, substituted or unsubstituted pyridinyl groups. A divalent cation may be chelated to a tertiary amine and one or more heteroaryl groups. Examples of divalent cations include, but are not limited to, Mn²⁺, Fe²⁺, Co²⁺, Ni²⁺, Cu²⁺, Zn²⁺, and the like. An end group may have the following structure:

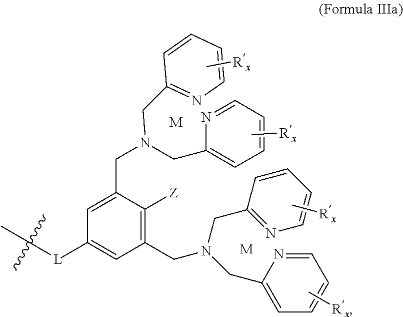

where L is O or —CH₂— and Z is OH, O, or H, where O is chelated to M, M is a divalent cation, R′ is independently at each occurrence chosen from hydrogen, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof, and x is 1, 2, 3, or 4. In various examples, an end group has the following structure:

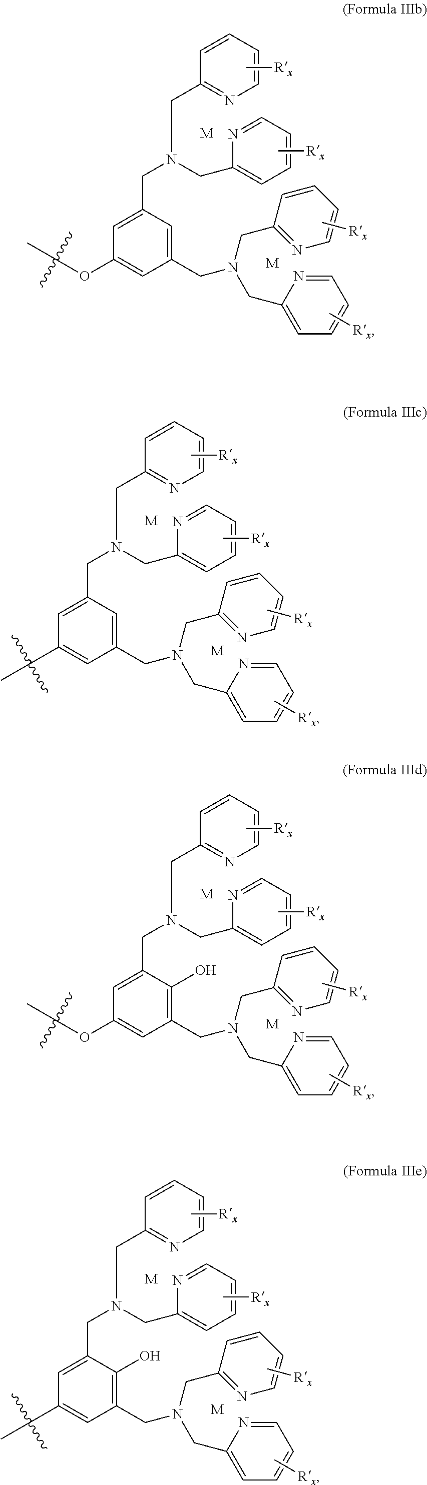

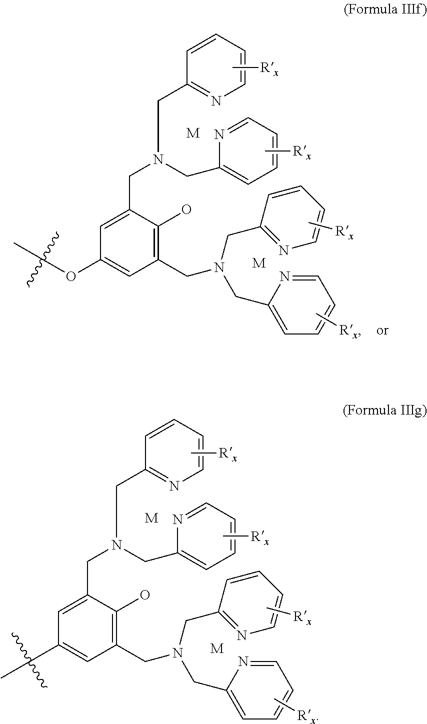

In various other examples, the end group has the following structure:

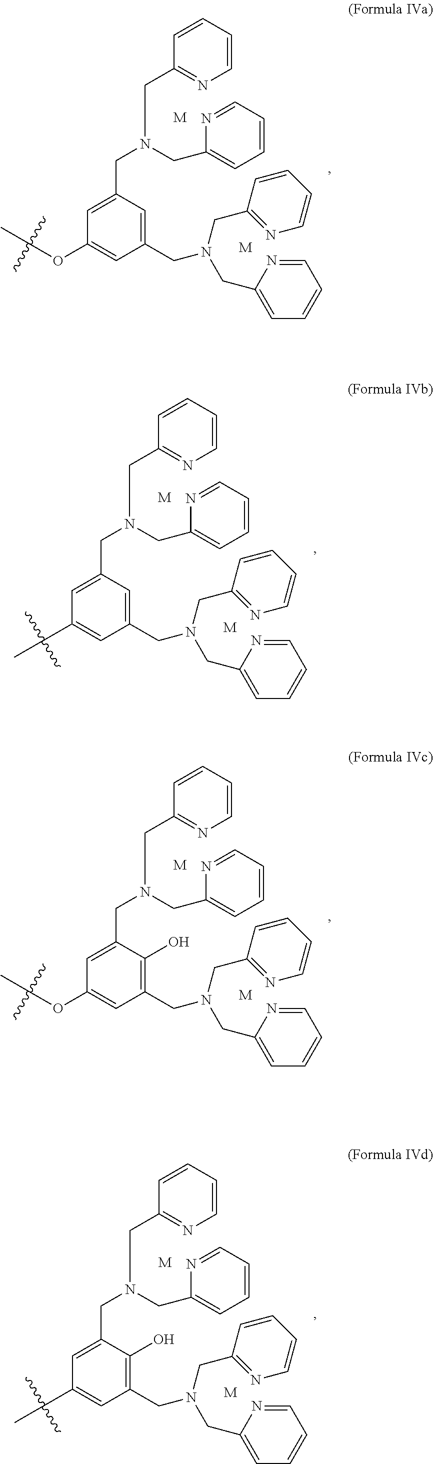

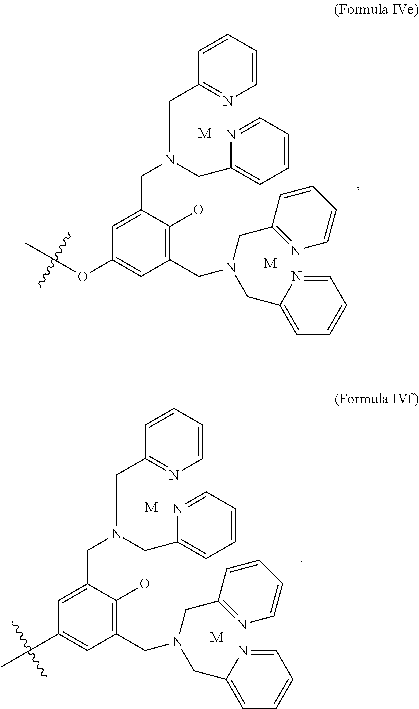

where M is a divalent cation, such as, for example, Zn²⁺.

In an aspect, the present disclosure provides compositions comprising one or more compounds of the present disclosure. The compositions may comprise one or more pharmaceutically acceptable carriers.

In an aspect, the present disclosure provides methods of using one or more compounds of the present disclosure. For example, the compounds can be used to treat an individual having cancer(s), one or more infectious diseases, chronic inflammation, and/or autoimmune conditions.

In an aspect, the disclosure provides kits. A kit may comprise pharmaceutical preparations containing any one or any combination of compounds and printed material.

### BRIEF DESCRIPTION OF THE FIGURES

For a fuller understanding of the nature and objects of the disclosure, reference should be made to the following detailed description taken in conjunction with the accompanying figures.

FIG. 1 shows a synthetic scheme to produce (ZnDPA)₆-DP-15K i.e., ExoBlock (9) with yields obtained for each step.

FIG. 2 shows structures of Zn-T-DPA (A) and ExoBlock (B). (C) ExoBlock inhibits exosome-mediated arrest of T cell activation. PBL were either unactivated (Unt) or activated for 2 h (hours) with immobilized antibodies to CD3 and CD28 with exosomes (Exo), exosomes with Zn-T-DPA and Exoblock (Exo+Zn-T-DPA and Exo+ExoBlock) or without (No Exo) exosomes. NFκB expression was detected using confocal microscopy.

FIG. 3 shows antigen-specific suppression of DM6 melanoma by TKT R438W cells is followed by tumor escape in the OTX model. (A) Engraftment of GFP+ tumor target cells DM6-WT and DM6-Mut into the omentum of NSG mice (B) TKT cells injected into mice 5 days following tumor injection significantly suppress the growth of DM6-Mut but not DM6-WT tumors (C) DM6-Mut tumors show recurrence following initial suppression. (D) Corrected total fluorescence was calculated using Image J. Mean±SEM **p>0.01 (E) Gross images of omenta on day 25.

FIG. 4 shows DM6 melanoma OTX growth kinetics. DM6 melanoma tumor cells transduced with a lentiviral expression system to express luciferase (DM6 Luc+) were injected i.p. into NSG mice (n=10). At various time points, luciferin substrate was injected i.p. and bioluminescence was measured. (A) Representative bioluminescence images of DM6 Luc+ tumor burden in mice from Day 3, 14 and 30. (B) DM6 Luc+ tumor growth in mice over time. (C) Adoptive transfer of TKT R438W T cells suppresses tumor growth of DM6-Mut tumors. Data shown as the arithmetic mean with error bars denoting SEM. *p≤0.05, ***p≤0.001.

FIG. 5 shows anti-PD-1 and liposomal IL-12 delay tumor escape in the X-mouse model. (A) Experimental scheme and timeline (B-C) Tumor burdens on respective days in the X-mouse model in anti-PD-1 experiment (B) or the IL-12 experiment (C). Corrected total fluorescence was calculated using Image J. Mean±SEM **p≤0.01.

FIG. 6 shows characterization of exosomes derived from DM6 Xenografts: (A) Size distribution analyzed by Nanoparticle tracking analysis (B) Exo Array showing the presence of exosomal markers (C) Presence of immunosuppressive lipids phosphatidylserine (PS) and ganglioside GD3 on exosomes attached to latex beads. Unstained (filled histogram), secondary antibody control (dashed line) and stained sample (solid line) are shown. (D) Exosomes inhibit T cell activation. PBL were either unactivated (Unact) or activated for 2 h with immobilized antibodies to CD3 and CD28 with (Act+Exo) or without (Act) exosomes. CD69 expression was detected by flow cytometry following overnight incubation.

FIG. 7 shows PD-L1 expression in DM6 cells and DM6 xenograft-derived exosomes. (A) PD-L1 expression in DM6-Mut cells cultured for 48 h without (U) or with (T) conditioned medium (from a 48 h co-culture of DM6-Mut cells with TKT R438W cells). (B) PD-L1 expression in ascites fluid-derived exosomes from mice with an untreated DM6-Mut Xenograft (1), a DM6-Mut Xenograft treated with TKT cells on day 5 (2).

FIG. 8 shows ExoBlock suppresses tumor growth and has comparable efficacy to anti-PD1 treatment in the X-mouse model. (A) Experimental scheme and timeline (B) Representative images of the omentum from various treatment groups on day 25 (C) Tumor burden represented as corrected total fluorescence calculated using Image J. The untreated group on day 25 had too much tumor to be accurately scanned. n=4-5 mice/group. Mean±SEM **p≤0.01.

FIG. 9 shows a synthetic scheme to prepare 6-arm Zn-T-DPA-DP-15K (13). Reagents: (i) Glutaric anhydride, CHCl3 (ii) EDC, DMF (iii) 6-ARM(DP)-NH2-15K (3) (iv) Zn(NO₃)₂.6H₂O.

FIG. 10 shows (A) experimental set up to determine the inhibitory effects of exosomes from ovarian ascites fluid with (Zn-DPA)₆-PEG. (B) Inhibitory effects of exosomes from ovarian ascites fluid with (Zn-DPA)₆-PEG.

FIG. 11 shows different batches of ExoBlock are consistent in their ability to reverse immunosuppressive effect of exosomes. T cells from normal donor peripheral blood leukocytes (NDPBL) were activated for 2 h with immobilized antibodies to CD3 and CD28 in the presence or absence of exosomes and 10 μM ExoBlock. The percentage of activation was determined by monitoring the upregulation of CD69. Percentage of inhibition and reversal were calculated.

FIG. 12 shows ExoBlock competitively inhibits binding of PSVue 499 to apoptotic cells in a dose-dependent manner. Jurkat cells were treated with 10 μM Etoposide for 20 h to induce apoptosis. The cells were then stained with PSVue with equimolar or titrating molar amounts of ExoBlock. Sytox Red was used to eliminate dead cells from the analysis. The experiment was done in triplicate wells. Representative data are shown in (A) and quantified data from 3 wells for equimolar amounts of ExoBlock is shown in (B). Dose-dependency of the competitive inhibition is shown in (C) and (D), highlighting the inverse relationship between ExoBlock dose and detection of PSVue fluorescence. The amount of fluorescence in resting cells is shown as baseline (21.3±5.7) for (C). Statistical analysis was done using unpaired Student's t test. ns=not significant; **p<0.01.

FIG. 13 shows NMR spectra of (A) a polymer arm precursor, (B) batch 1 of ExoBlock, (C) batch 2 ExoBlock, (D) batch 3 ExoBlock, (E) batch 4 ExoBlock, and (F) batch 5 ExoBlock.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

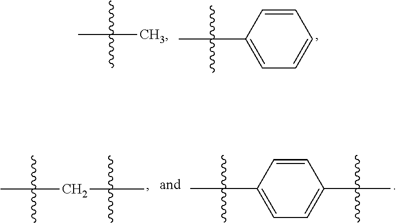

As used herein, unless otherwise indicated, the term “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₃₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C22, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, the term “heteroaryl” refers to a C₁ to C₁₄ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, and C₁₄). The heteroaryl groups may be substituted or unsubstituted. Examples of heteroaromatic groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof.

The present disclosure provides compounds that bind phosphatidylserine (PS). Also provided are compositions comprising the compounds and methods of using the compounds and/or compositions.

In an aspect, the present disclosure provides compounds comprising a branching group having the following structure:

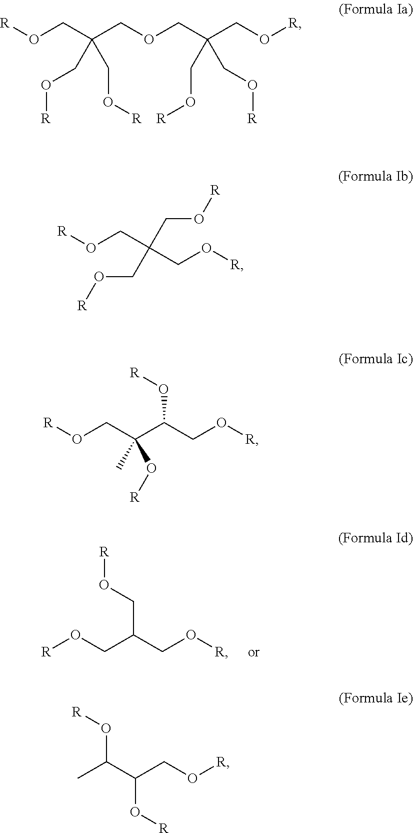

where each R is independently at each occurrence hydrogen or comprises a poly(ethylene glycol) (PEG) group or an ethylene glycol group, a linker group, and an end group. The compounds may also have various counter anions. One or more of the R groups may be the same or different. In various examples, one or more of the R groups are hydrogen (e.g., for Formula Ia: one, two, three, four, or five R groups may be hydrogen; for Formula Ib and Ic: one, two, or three R groups may be hydrogen, for Formulas Id and Ie: one or two R groups may be hydrogen).

A PEG group may have various lengths. The PEG group may have 2-500 repeat units, including every integer value and range therebetween. In various examples, the molecular weight (e.g., Mn) of the PEG group may be 2,000-60,000, including every integer value and range therebetween (e.g., 8,000-15,000).

A linker group is connected (e.g., covalently bonded) to the PEG group or ethylene glycol group at one end and is connected to the end group at the other end. The linker group may have the following structure:

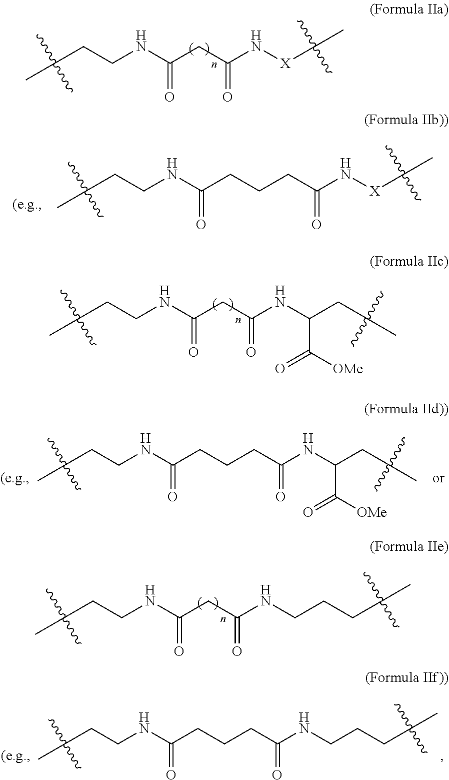

where X is a spacer group, such as, for example, a substituted or unsubstituted C₁ to C₁₀ alkyl group and n is 2, 3, or 4.

An end group comprises various aryl groups, heteroaryl groups, a tertiary amine, and a plurality of divalent cations. The heteroaryl groups may have various substituents, such as, for example, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof. One, some, or all of the heteroaryl groups may be, for example, substituted or unsubstituted pyridinyl groups. A divalent cation may be chelated to a tertiary amine and one or more heteroaryl groups. Examples of divalent cations include, but are not limited to, Mn²⁺, Fe²⁺, Co²⁺, Ni²⁺, Cu²⁺, Zn²⁺, and the like. An end group may have the following structure:

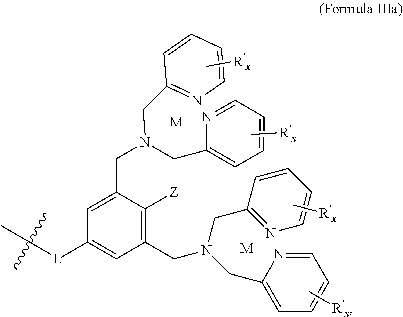

where L is O or —CH₂— and Z is OH, O, or H, where O is chelated to M, M is a divalent cation, R′ is independently at each occurrence chosen from hydrogen, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof, and x is 1, 2, 3, or 4. In various examples, an end group has the following structure:

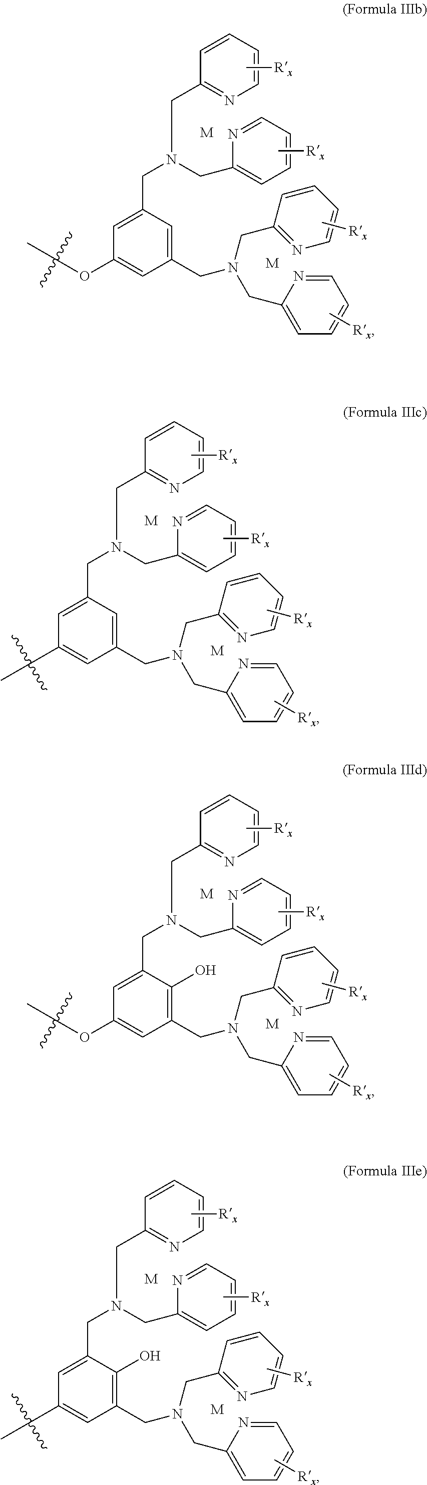

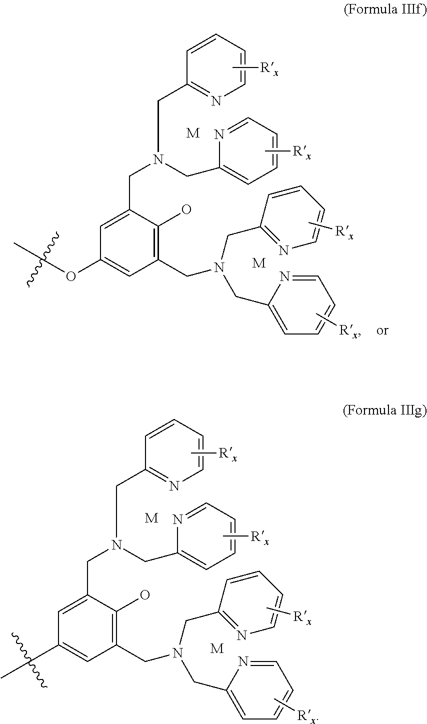

In various other examples, the end group has the following structure:

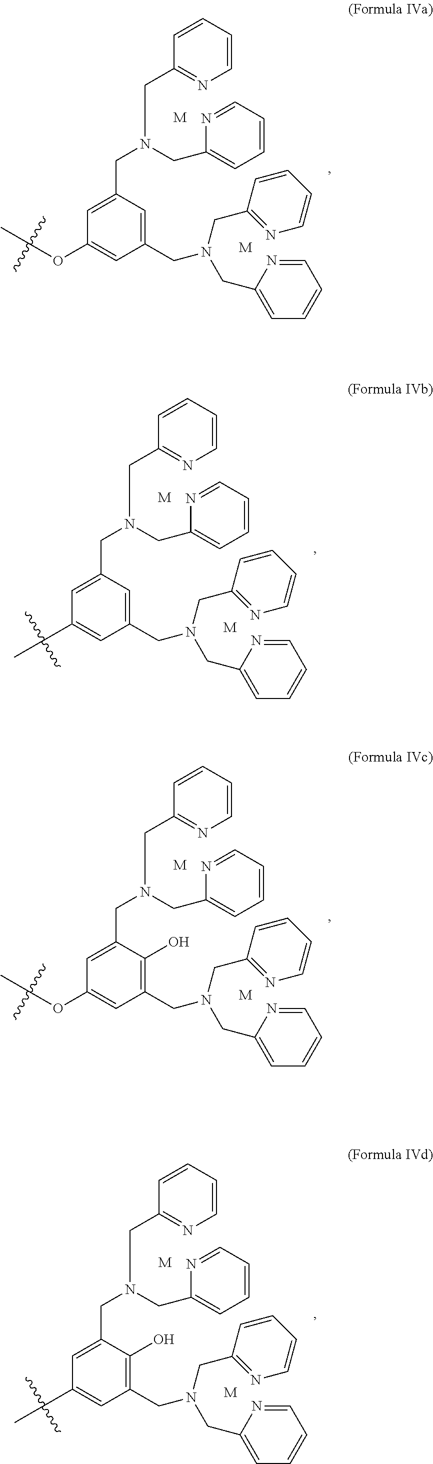

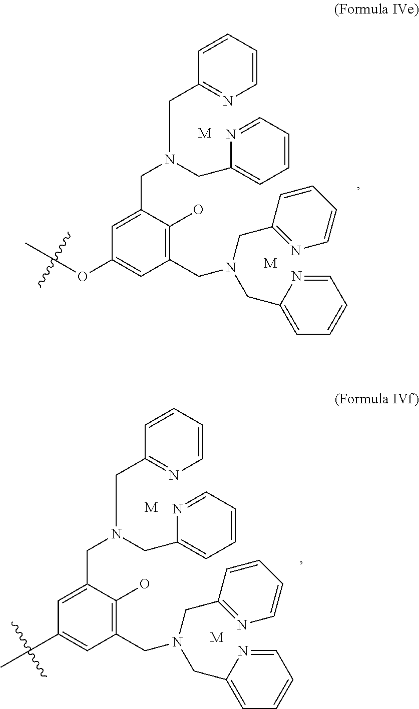

where M is a divalent cation, such as, for example, Zn²⁺.

In various examples, a compound of the present disclosure may have the following structure:

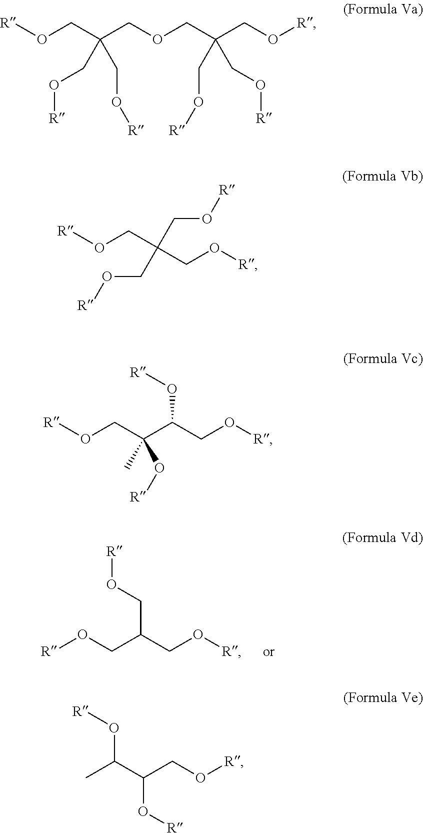

where R″ is independently at each occurrence H or

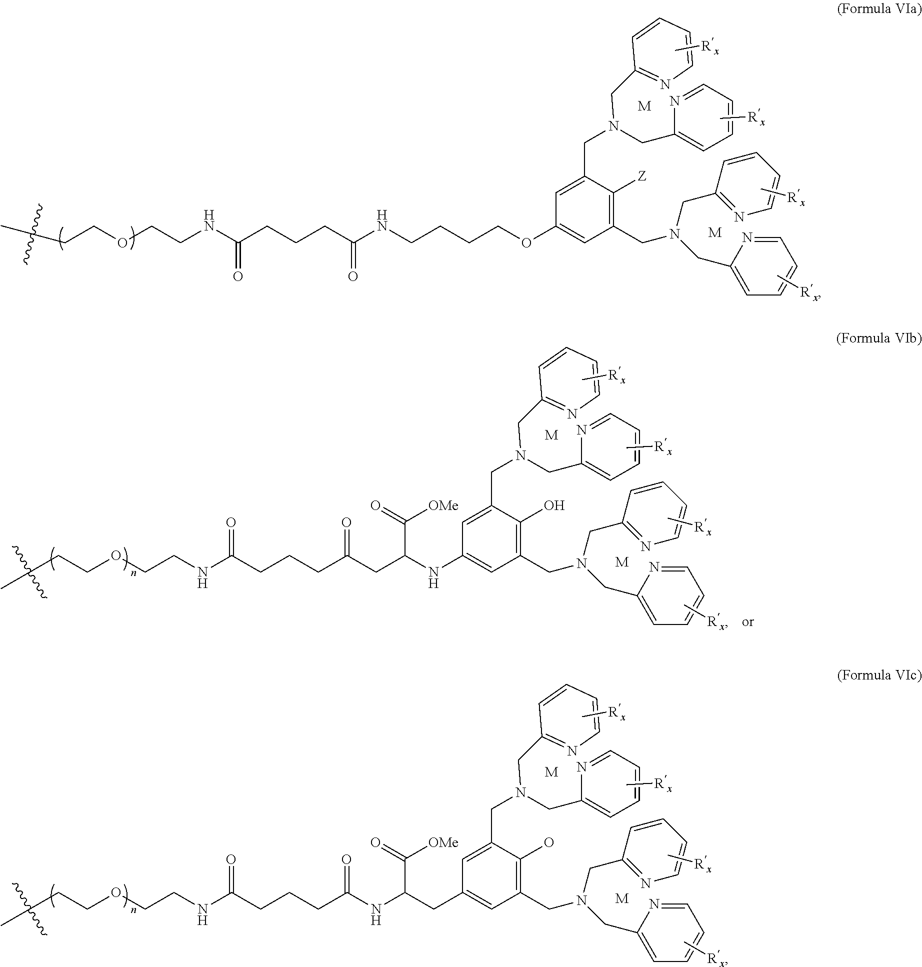

where M is a divalent cation, R′ is independently at each occurrence chosen from halogens |(—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof, A is one or more counter anions (e.g., NO₃⁻, CH₃CO₂⁻, SO₄²⁻, the like, and combinations thereof), x is 1, 2, 3, or 4, and n is 1-500, including every integer value and range therebetween.

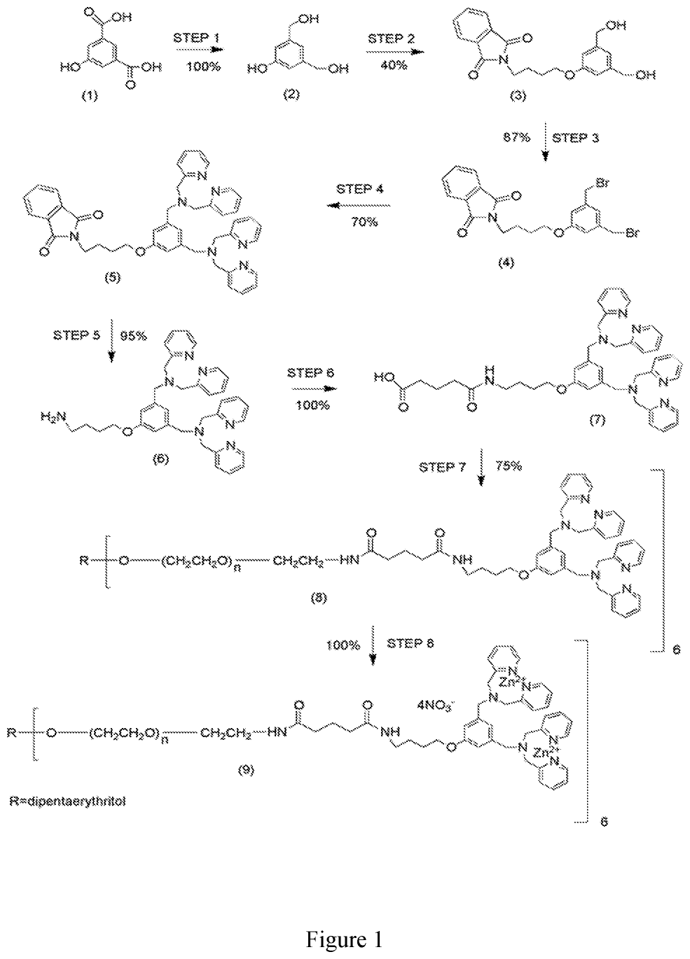

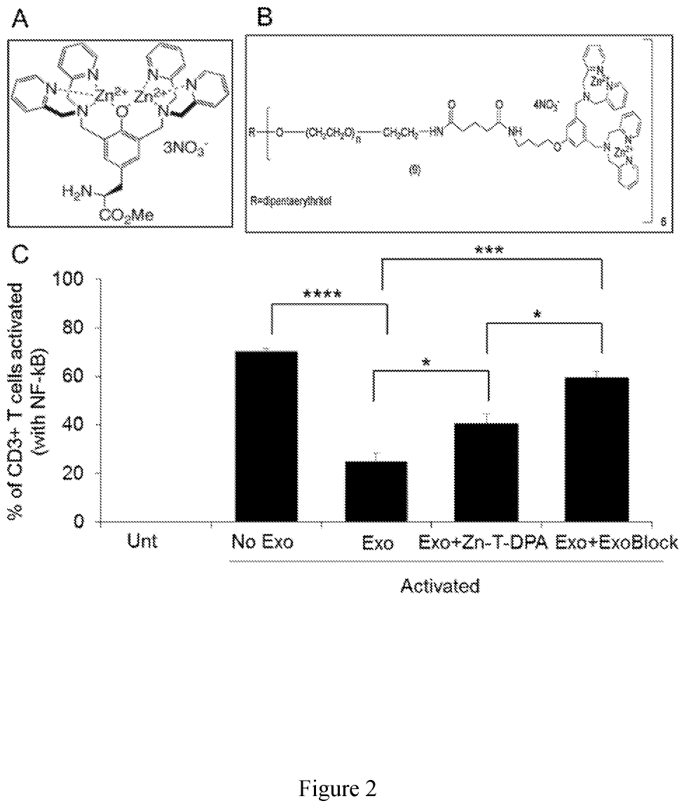

A compound of the present disclosure may have the following structure:

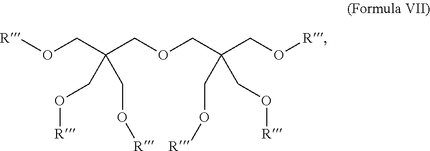

where R″′ is

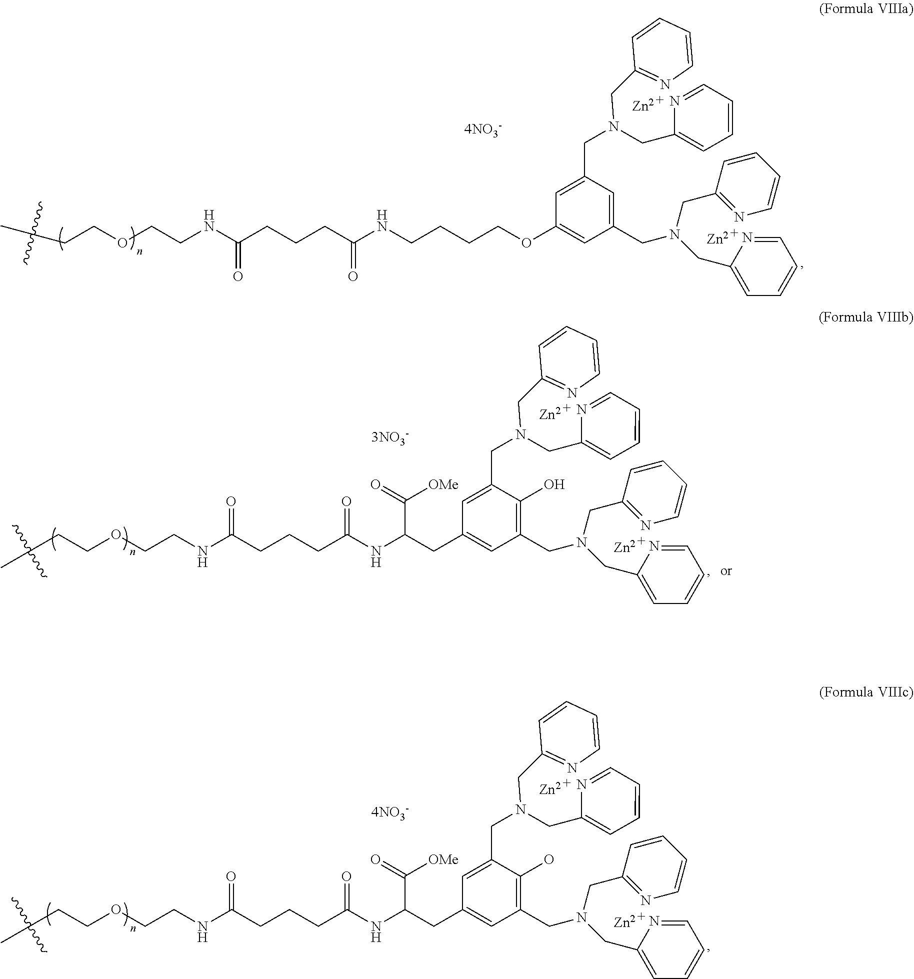

where n is 1-500, including every integer value and range therebetween. A compound having this structure may bind 2-24 PS molecules, including every integer value and range therebetween. In various examples, this structure may bind 2-12, 2-10, 2-8, or 2-6 PS compounds (e.g., 2, 3, 4, 5, 6, 7, 8, 9, 10, 11, or 12). Without intending to be bound by any particular theory, binding of PS molecules may depend on the local concentration. A compound having the structure of Formula VII, where R″′ is Formula VIIIa may be referred to as “ExoBlock.” See FIGS. 1 and 2.

In an aspect, the present disclosure provides compositions comprising one or more compounds of the present disclosure. The compositions may comprise one or more pharmaceutically acceptable carriers.

In an embodiment, the compounds of the present disclosure may be provided in delivery vehicles, such as, for example, liposomes, polylactic acid microspheres, nanoparticles (e.g., latex beads, exosomes, polylactic co-glycolic acid nanoparticles (PLGA nanoparticles) and the like), and the like. In various examples, the liposomes may incorporate one or more compounds of the present disclosure. The liposome monolayer or bilayer may comprise phosphatidylcholine (“PC”) and/or phosphatidylglycerol (“PG”) and, optionally, cholesterol. PG and PC may have 2-22 carbon atoms in the acyl chain. In one embodiment, the acyl chains have 2 to 22 or 6 to 22 carbons, including all integer number of carbons and ranges therebetween. The acyl chains may be saturated or unsaturated and may be same or different lengths. Some examples of acyl chains are: lauric acid, myristic acid, palmitic acid, stearic acid, arachidic acid, behenic acid, oleic acid, palmitoleic acid, linoleic acid, and arachidonic acid. The PG or PC can have one or two acyl chains. In various examples, the phospholipids are present in a ratio of 10:90, 20:80, 30:70, 40:60, 50:50, 60:40, 70:30, 80:20, or 90:10 PG to PC. In various examples, the size of 50, 60, 70, 80, 90, 95 or 100% (including all percentages between 50 and 100) of the liposomes is 40 nm to 4 μm, including all sizes therebetween in the nanometer and micrometer range. In various examples, the liposomes may be multilamellar.

The compositions described herein may include one or more standard pharmaceutically acceptable carriers. Pharmaceutically acceptable carriers may be determined in part by the particular composition being administered, as well as by the particular method used to administer the composition. Accordingly, there are a wide variety of suitable formulations of pharmaceutical compositions of the present disclosure. The compounds may be freely suspended in a pharmaceutically acceptable carrier or the compounds may be encapsulated in liposomes and then suspended in a pharmaceutically acceptable carrier. Examples of carriers include solutions, suspensions, emulsions, solid injectable compositions that are dissolved or suspended in a solvent before use, and the like. Compositions (e.g., injections and the like) may be prepared by dissolving, suspending or emulsifying one or more of the active ingredients in a diluent. Examples of diluents, include, but are not limited to distilled water for injection, physiological saline, vegetable oil, alcohol, dimethyl sulfoxide, and the like, and combinations thereof. Further, the injections may contain stabilizers, solubilizers, suspending agents, emulsifiers, soothing agents, buffers, preservatives, and the like, and combinations thereof. Compositions (e.g., injections and the like) may be sterilized in a formulation step or prepared by sterile procedure. A composition may be formulated into a sterile solid preparation, for example, by freeze-drying, and can be used after sterilized or dissolved in sterile injectable water or other sterile diluent(s) before use (e.g., immediately before use). Additional examples of pharmaceutically include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, Pa. Lippincott Williams & Wilkins. Effective formulations include, but are not limited to, oral and nasal formulations, formulations for parenteral administration, and compositions formulated for with extended release. Parenteral administration includes infusions such as, for example, intramuscular, intravenous, intraarterial, intraperitoneal, subcutaneous administration, and the like.

Examples of compositions include, but are not limited to, (a) liquid solutions, such as, for example, an effective amount of a compound of the present disclosure suspended in diluents, such as, for example, water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The liquid solutions described above may be sterile solutions. The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

A composition may be in unit dosage form. In such form, the composition may be subdivided into unit doses containing appropriate quantities of the active component. The unit dosage form may be a packaged preparation, the package containing discrete quantities of preparation, such as, for example, packeted tablets, capsules, and powders in vials or ampoules. Also, the unit dosage form may be a capsule, tablet, cachet, or lozenge itself, or it may be the appropriate number of any of these in packaged form. The composition can, if desired, also contain other compatible therapeutic agents. The compositions may deliver the compounds of the disclosure in a sustained release formulation.

In an aspect, the present disclosure provides methods of using one or more compounds of the present disclosure. For example, the compounds can be used to treat an individual having cancer(s), one or more infectious diseases, chronic inflammation and chronic inflammation diseases, and/or autoimmune conditions.

Examples of infectious diseases include, but are not limited to, bacterial diseases, viral diseases, parasitic diseases, and the like, and combinations thereof. Examples of chronic inflammatory diseases include, but are not limited to, chronic rhinosinusitis with nasal polyposis, atopy, hepatitis, and the like, and combinations thereof. Examples of autoimmune diseases include, but are not limited to, rheumatoid arthritis, systemic lupus, erythematosus, diabetes, and the like, and combinations thereof.

A method of treating may comprise administering to an individual one or more compounds of the present disclosure or a composition comprising one or more compounds of the present disclosure. In various examples, a composition comprises one or more compounds and a checkpoint inhibitor (e.g., an anti-PD1 antibody, such as, for example, nivolumab, pembrolizumab, durvalumab, camrelizumab, cemiplimab, sintilimab, toripalimab, or the like, or a combination thereof). Additional examples of checkpoint inhibitors include, but are not limited to, anti-CTLA-4 antibodies, anti-LAG3 antibodies, anti-TIM3 antibodies, and the like, and combinations thereof. The composition may comprise or further comprise immunotherapeutics, such as, for example, cytokines (e.g., IL-12, IL-2, and the like, and combinations thereof, for modulating immune response.

The method may be carried out in an individual who has been diagnosed with or is suspected of having cancer (e.g., a solid tumor (such as, for example, a solid tumor associated with melanoma), leukemia, lymphoma, and the like, and combinations thereof).

In various examples, compounds and/or compositions of the present disclosure are more effective than Zn-T-DPA, which is depicted in FIG. 2A.

Compositions comprising the compounds described herein may be administered to an individual using any known method and route, including oral, parenteral, subcutaneous, intraperitoneal, intrapulmonary, intranasal, and intracranial injections. Parenteral infusions include, but are not limited to, intramuscular, intravenous, intraarterial, intraperitoneal, subcutaneous administration, and the like. Administration may also include, but is not limited to, topical and/or transdermal administrations.

The dose of the composition comprising a compound of the present disclosure and a pharmaceutical agent may necessarily be dependent upon the needs of the individual to whom the composition of the disclosure is to be administered. These factors include, for example, the weight, age, sex, medical history, and nature and stage of the disease for which a therapeutic or prophylactic effect is desired. The compositions may be used in conjunction with any other conventional treatment modality designed to improve the disorder for which a desired therapeutic or prophylactic effect is intended, non-limiting examples of which include, but are not limited to, chemotherapy, surgical interventions and radiation therapies. For example, the compositions are used in combination with (e.g., co-administered with) one or more known anti-cancer drugs (e.g., DNA damaging anti-cancer drugs).

Compounds and compositions comprising compounds may be dosed at various dosages. Examples include, but are not limited to, 1 to 300 mg/kg, including every 0.1 mg/kg value and range therebetween. In various examples, a dose may be 1-100 mg/kg, 1-200 mg/kg, 2-200 mg/kg, 2-300 mg/kg, 5-100 mg/kg, 5-200 mg/kg, 5-300 mg/kg, 40-80 mg/kg, 50-70 mg/kg, 50-100 mg/kg, 50-150 mg/kg, 50-200 mg/kg, 50-250 mg/kg, 50-300 mg/kg, 55-70 mg/kg, 25-100 mg/kg, 25-200 mg/kg, 25-300 mg/kg, 100-200 mg/kg, 100-300 mg/kg, 150-200 mg/kg, 150-300 mg/kg, 200-250 mg/kg, or 200-300 mg/kg.

In an aspect, the disclosure provides kits. A kit may comprise pharmaceutical preparations containing any one or any combination of compounds and printed material.

In various examples, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information. The printed information may be provided on a label, or on a paper insert, or printed on the packaging material itself. The printed information may include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material may include an indication that the pharmaceutical composition and/or any other agent provided with it is for treatment of a subject having cancer and/or other diseases and/or any disorder associated with cancer and/or other diseases. In various examples, the product includes a label describing the contents of the container and providing indications and/or instructions regarding use of the contents of the container to treat a subject having cancer(s), one or more infectious diseases, chronic inflammation, and/or autoimmune conditions. A kit may comprise a single dose or multiple doses.

Methods of the present disclosure may be used on various individuals. In various examples, an individual is a human or non-human mammal. Examples of non-human mammals include, but are not limited to, farm animals, such as, for example, cows, hogs, sheep, and the like, as well as service, pet, and/or sport animals such as, for example, horses, dogs, cats, and the like. Additional non-limiting examples of individuals include, but are not limited to, rabbits, rats, mice, and the like. The compounds or compositions of the present disclosure may be administered to individuals, for example, in pharmaceutically-acceptable carriers, which may facilitate transporting the compounds from one organ or portion of the body to another organ or portion of the body.

The following Statements describe various embodiments of the present disclosure.

Statement 1. A compound of comprising a branching group having the following structure:

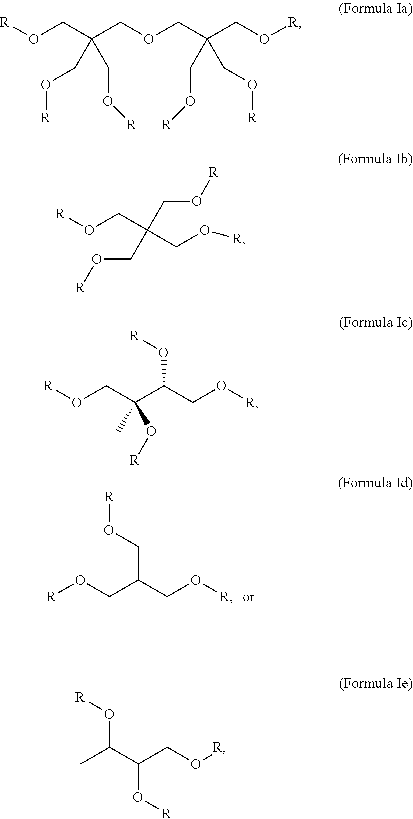

where each R is independently at each occurrence hydrogen or comprises a poly(ethylene glycol) (PEG) group or an ethylene glycol group, a linker group, and an end group.
Statement 2. A compound according to Statement 1, where the linker group has the following structure:

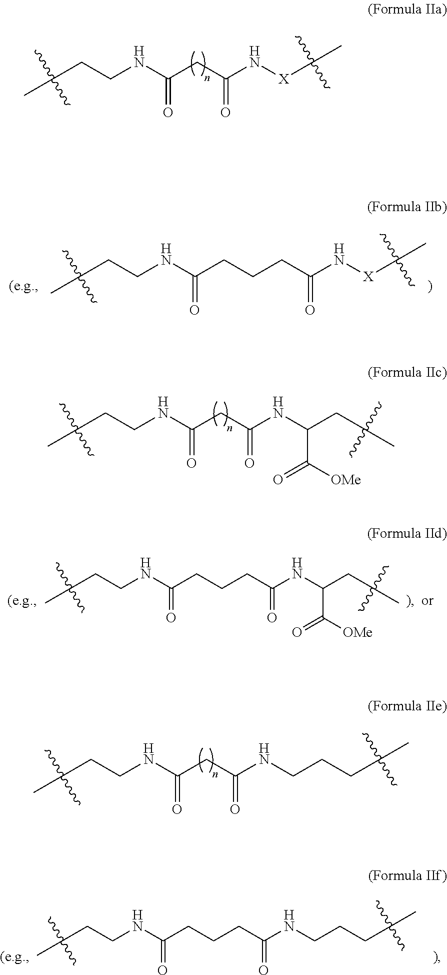

where X is a spacer group (e.g., a substituted or unsubstituted C₁ to C₁₀ alkyl group).
Statement 3. A compound according to Statement 1 or Statement 2, where the end group has the following structure:

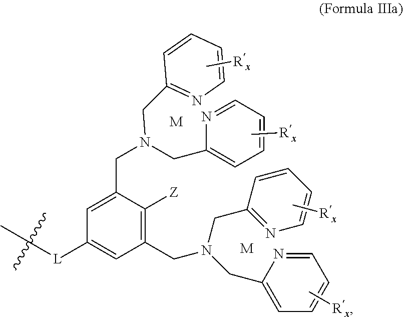

where L is O or —CH₂— and Z is OH, O, or H, where O is chelated to M, R′ is independently at each occurrence chosen from hydrogen, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof, and x is 1, 2, 3, or 4.
Statement 4. A compound according to Statement 3, where the end group has the following structure:

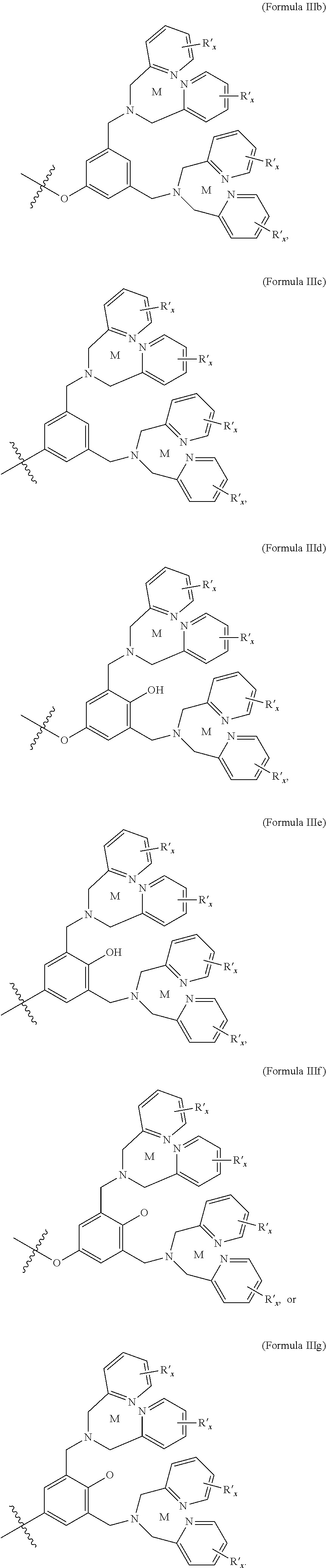

Statement 5. A compound according to Statement 3 or Statement 4, where the end group has the following structure:

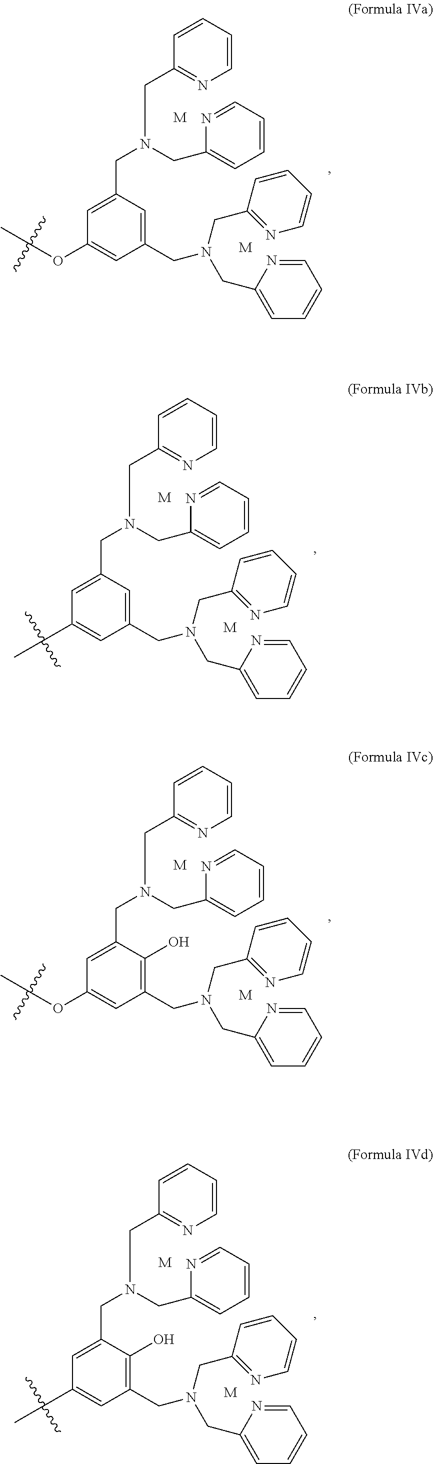

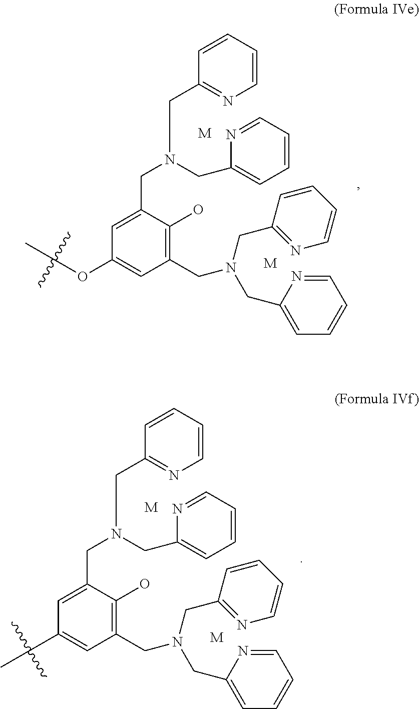

Statement 6. A compound according to any one of the preceding Statements, where the compound has the following structure:

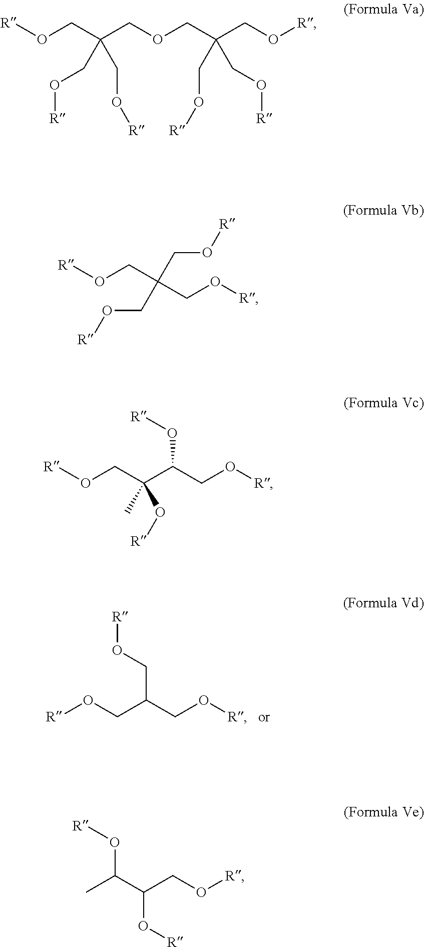

where R″ is independently at each occurrence H or

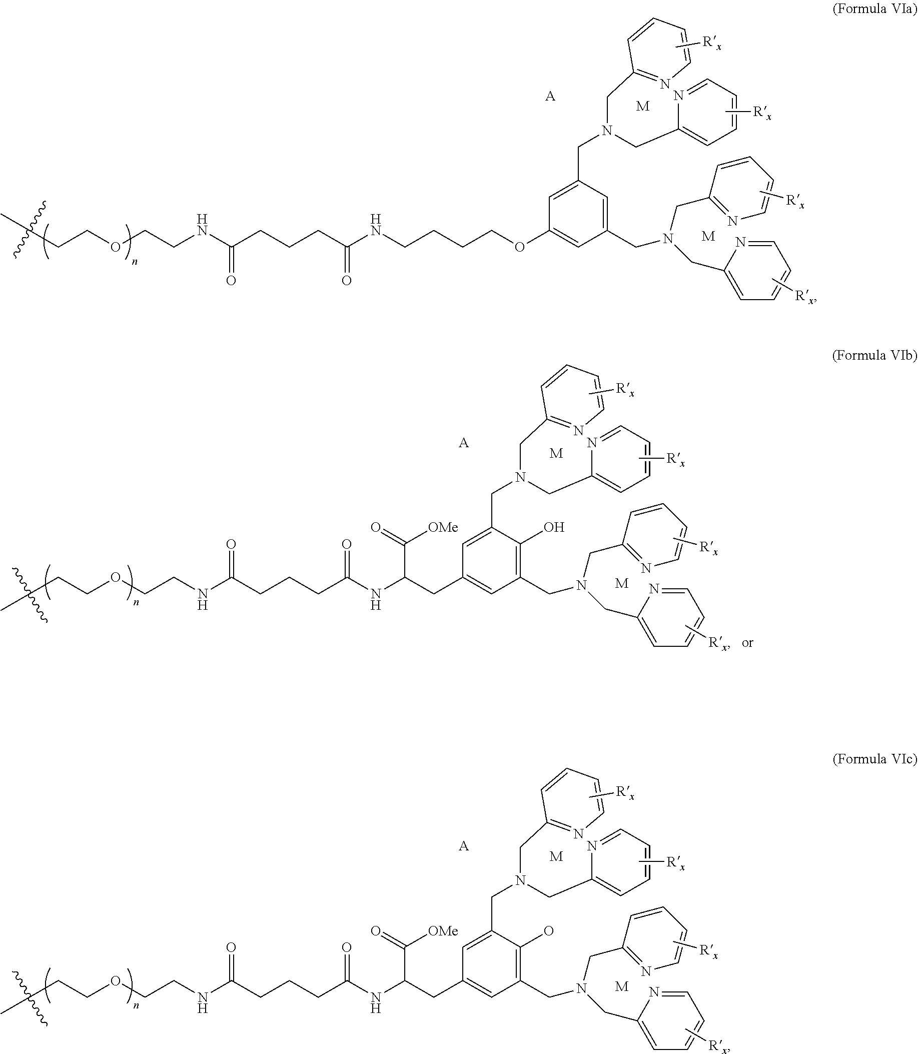

where M is a divalent cation, R′ is independently at each occurrence chosen from halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof. A is one or more counter anions (e.g., NO₃⁻, CH₃CO₂⁻, SO₄²⁻, the like, and combinations thereof), x is 1, 2, 3, or 4, and n is 1-500, including every integer value and range therebetween.
Statement 7. A compound according to Statement 6, where the compound has the following structure:

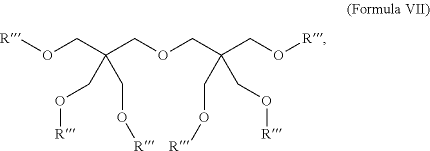

where R″′ is independently at each occurrence H or

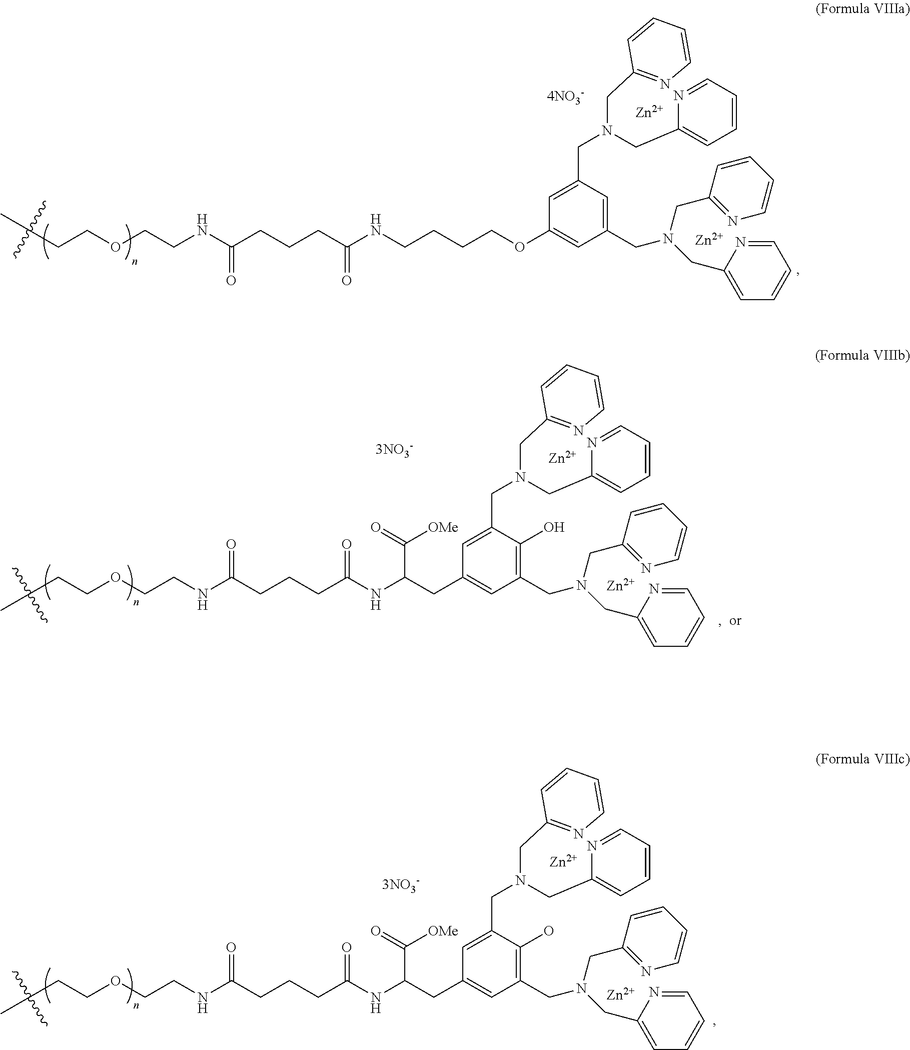

wherein n is 1-500, including every integer value and range therebetween.
Statement 8. A composition comprising a compound of the present disclosure (e.g., according to any one of the preceding Statements) and one or more pharmaceutically acceptable carriers.
Statement 9. A composition according to Statement 8, further comprising anti-PD1 antibodies (e.g., anti-PD1 antibodies chosen from nivolumab, pembrolizumab, durvalumab, camrelizumab, cemiplimab, sintilimab, toripalimab, and the like, and combinations thereof), anti-CTLA-4 antibodies, anti-LAG3 antibodies, anti-TIM3 antibodies, and the like, and combinations thereof.
Statement 10. A liposome composition, where the liposomes have incorporated therein a compound according to any one of Statements 1-7.
Statement 11. A liposome composition according to Statement 10, where the liposome has a monolayer or bilayer and the monolayer or bilayer comprise phosphatidylcholine (“PC”) and/or phosphatidylglycerol (“PG”) and, optionally, cholesterol.
Statement 12. A method of treating an individual in need of treatment (e.g., a human or non-human mammal) for cancer (e.g., a solid tumor (such as, for example, a solid tumor associated with melanoma), leukemia, lymphoma, and the like, and combinations thereof), one or more infectious diseases, chronic inflammation, and/or autoimmune conditions, comprising administering to the individual one or more compounds of the present disclosure (e.g., a compound according to any one of Statements 1-7) or one or more composition of the present disclosure (e.g., a composition according to any one of Statements 8-10).

The following examples are presented to illustrate the present disclosure. They are not intended to be limiting in any matter.

### EXAMPLE 1

The following in an example of synthesis and use of a compound of the present disclosure.

Design, synthesis, and testing of a new PS binding molecule ExoBlock that binds to tumor associated exosomes and blocks their ability to arrest T cell function.

Strategies to block PS in cancer and infectious diseases in preclinical studies using anti-PS antibodies and annexin V, or to treat lung cancer in clinical trials using a PS specific antibody (bavituximab) have met with modest success owing to relatively low affinity PS-binding of the molecules used. ExoBlock represents an exosome blocking molecule. ExoBlock is a hexamer that has been engineered to carry six binding sites for PS, which is more than an antibody or Annexin V and hence expected to bind PS with a much higher avidity. It was determined that ExoBlock does bind PS with a high avidity and is more effective than both anti-PS antibody and annexin V in blocking exosomal immune suppression in vitro. The therapeutic efficacy of ExoBlock in vivo has been established pre-clinically using a new animal model.

Design and validation of a novel animal model to establish efficacy of exosome blocking drugs.

The animal tumor xenograft model is a platform that uses patient-derived tumor-specific T cells to successfully and pre-clinically test the efficacy of immune based therapies for human cancer. This model uses T cells that are specific for neo-antigen peptides expressed on human tumor target cells in the context of HLA Class 1. This model is described, and the data generated using the model are presented herein.

Synthesis of the Zn compound with multiple phosphatidylserine (PS) binding sites that were determined to block exosome T cell immune suppression more effectively than compound Zn-T-DPA.

ExoBlock [(ZnDPA)₆-DP-15K] was synthesized, which has multiple binding sites and greater avidity to PS as compared to Zn-T-DPA. ExoBlock was synthesized at the 0.5 g scale via 8 synthetic steps (FIG. 1) with an overall synthetic yield of ˜18%. The penultimate product (minus zinc ions) was purified by a dialysis process and then eventually lyophilized to produce ExoBlock.

Step 1: Reaction between commercially available, dimethyl-5-hydroxy-isophthalate and lithium aluminum hydride in tetrahydrofuran at reflux for 24 hours (h) produced the triol (2). Step 2: The reaction between (2) and N-(4-bromobutyl)phthalamide was performed by heating the 2 compounds together overnight in acetonitrile in the presence of potassium carbonate. Step 3: Bromination of (3) with carbon tetrabromide and triphenylphosphine followed by chromatographic purification worked well on the 1-2 g scale to produce (4) in high yield. Step 4: This reaction proceeded in good yield on the small scale (1-2 g) by vigorously stirring (4) with 2 mole equivalents di-(2-picolyl)-amine in N,N-dimethylformamide containing potassium carbonate for 24 h. Product (5) was purified by normal phase silica gel chromatography using dichloromethane/methanol mixtures containing ammonium hydroxide. Step 5: Reaction to remove the phthalimido protecting group from intermediate (5) was performed by refluxing with concentrated hydrochloric acid and took 48 h for complete reaction. Step 6: Reaction of (6) with glutaric anhydride in chloroform overnight provided (7) in quantitative yield with no further purification performed. Step 7: The sulfosuccinimide ester of (7) was formed in situ upon reaction with a water soluble carbodiimide (EDC) and N-hydroxysulfosuccinimide, and then an excess of this activated ester mixture was added to the 6-arm-PEG-amino functionalized polymer (MW=15K) in DMF. After stirring overnight the mixture was dialyzed (MWCO=8-10K) against water and the resulting solution lyophilized to provide (8). Step 8: This transformation was affected in quantitative yield by treating (8) with an aqueous solution of 12 mole equivalents of zinc nitrate followed by lyophilization.

ExoBlock reversed the exosome-mediated arrest of T cell function with a greater efficacy (75-96% reversal), than the compound Zn-T-DPA (30-45% reversal) in comparative studies (FIG. 2A-C). These studies have been repeated for different endpoints of activation such as nuclear translocation of NFκB and intracellular cytokine expression, and the efficacy of ExoBlock has been highly reproducible across assays.

### Toxicity Studies of ExoBlock

A systemic organ toxicity study at three relatively high doses of Zn-T-DPA (i.e., 2, 10 and 50 mg/Kg) was shown to have No Observed Adverse Effect Level (NOAEL) in mice. The original PK studies for this drug were previously initiated, but these studies were terminated when after ExoBlock was synthesized.

Systemic organ toxicity study was completed in mice with ExoBlock at a dose of 64 mg/kg. NOAEL was observed at the dose and schedule for ExoBlock. Mice were euthanized 14 days after treatment with the drugs. Selected organs from mice treated with ExoBlock and the control untreated mice were removed, fixed, sectioned, stained and examined microscopically for the evidence for histopathology. No pathology was observed in the organs examined in lung, spleen, small intestine, kidney, or liver. A more complete and robust systemic organ toxicity will be done in mice and non-human primates. The PK studies are outlined herein to establish drug bioavailability and to monitor for possible off target drug effects. These studies will evaluate the efficacy of ExoBlock used in a soluble form or encapsulated into liposomes.

The X mouse model was established to allow for the rapid generation of human tumors and an in vivo method of evaluation of the anti-tumor responses of patient-derived T cells to patient tumor-specific peptides expressed by the established tumors. This model can easily enable preclinical testing of the efficacy of personalized immune based therapies, non-personalized immune based therapies, and many other anti-cancer therapies either alone or in combination.

There are 7 different T cells derived for 3 different patients available for our studies (Table 1). Using cell sorting, the anti-tumor T cells have been purified to about 95-99% antigen-specificity. These T cells are activated specifically by peptides presented in the context of HLA-A*02:01 by melanoma tumor target cells. The melanoma tumor target cells (DM6) are either transduced with a tandem mini-gene expressing GFP or with luciferase and each are genetically modified to express either the mutated peptide (DM6-Mut), tumor target or the wild type peptide (DM6-WT) control target. Tumor growth in the X mouse model is monitored either by post-mortem quantification of GFP fluorescence in the omentum or by quantification of luciferase-dependent bioluminescence of the mice by live imaging.

TABLE 1

| Patient | T cells | Mutated Peptide Recognized |
|---|---|---|
| Mel 21 | TKT R438W | AMFWSVPTV (SEQ ID NO: 1) |
| | TMEM48 F169L | CLNEYHLFL (SEQ ID NO: 2) |
| | CDKN2A E153K | KMIGNHLWV (SEQ ID NO: 3) |
| Mel 38 | SEC24A P469L | FLYNLLTRV (SEQ ID NO: 4) |
| | AKAP13 Q285K | KLMNIQQKL (SEQ ID NO: 5) |
| Mel 218 | EXOC8 Q656P | IILVAVPHV (SEQ ID NO: 6) |
| | PABPC1 R520Q | MLGEQLFPL (SEQ ID NO: 7) |

Tumor-associated immune suppressive exosomes released from DM6-Mut tumor cells are present in the microenvironment of tumor xenografts in the X-mouse model.

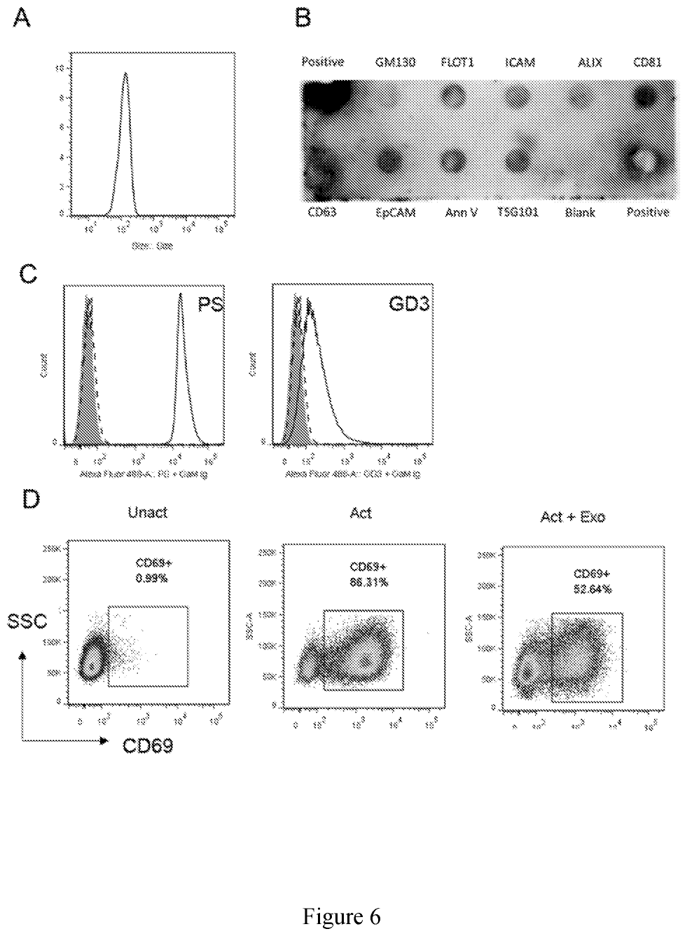

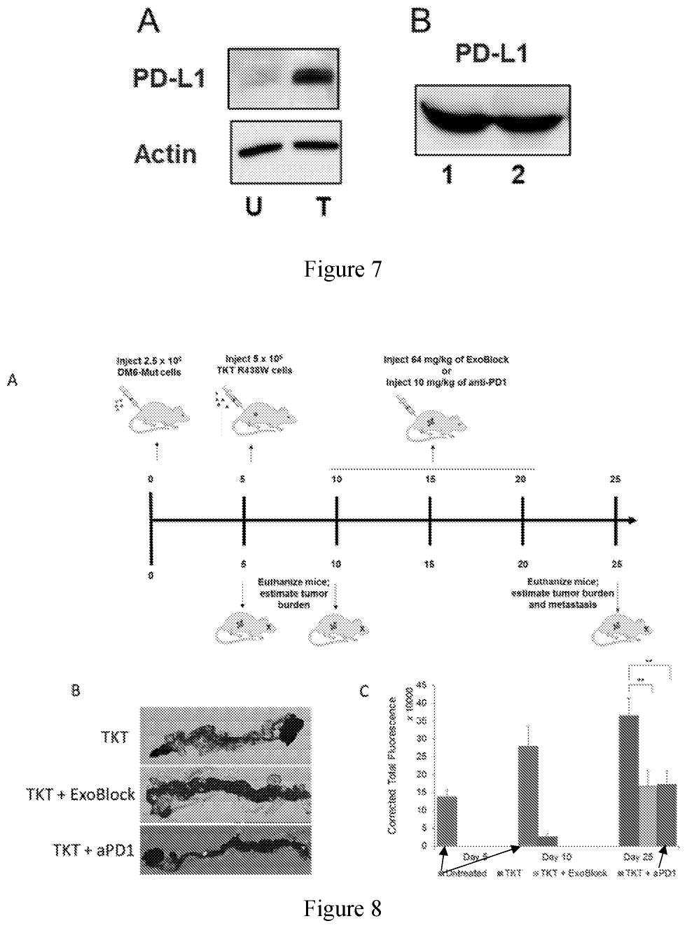

The presence of exosomes in the tumor xenografts and that they inhibit the activation of T cells was demonstrated. Without intending to be bound by any particular theory, it is believed ExoBlock is acting to suppress the exosomes, enhance the T cell anti-tumor activity and delay the tumor escape in the mouse model. Extracellular vesicles have been isolated from DM6-Mut melanoma tumor xenografts using methods previously reported (Keller et al., Cancer Immunol. Res., 2015, 3(11): 1269-78). Based upon size (125-150 nm) and composition (CD63, CD81, FLOT1, and ALIX) these melanoma-associated extracellular vesicles have been identified as exosomes and they are immunosuppressive (FIGS. 6A, B and D). These tumor associated exosomes also express the lipids that our exosome blocking drugs are ExoBlock is targeting, PS and GD3 (FIG. 6C). Additionally, western blot analysis showed that DM6-Mut tumor cells upregulate PD-L1 expression when cultured in conditioned medium from activated TKT cells (FIG. 7A). PD-L1 is also expressed on the exosomes isolated from ascites fluids of DM6-Mut tumor bearing mice and solid DM6-Mut tumor xenografts (FIG. 7B), which is consistent with data suggesting that tumors in melanoma patients do shed PD-L1+ exosomes that suppress tumor specific T cells and are associated with tumor growth and progression.

X-mouse model establishes the in vivo efficacy of ExoBlock.

The X-mouse model was used to test the efficacy of ExoBlock. ExoBlock was injected i.p. into NSG mice bearing DM6-Mut tumor xenografts and treated with TKT cells. The dose of ExoBlock (64 mg/kg of) was determined based upon the concentration that was determined to block the exosome mediated T cell suppression in vitro. It was found that at the dose tested (64 mg/kg), ExoBlock significantly delayed tumor escape (two-fold change in tumor burden on day 25), which was comparable to treatment with anti-PD1 (nivolumab at 10 mg/kg) (FIG. 8). These data establish that the efficacy of ExoBlock and confirms the viability of approaches to target immunosuppressive exosomes in tumor microenvironments.

It was established in these pre-clinical efficacy studies that ExoBlock has no detectable toxicities and it does not interfere with the anti-tumor responses of the tumor-specific T cells in the mouse model. In vitro studies have also established that ExoBlock does not directly kill the tumor target cells (DM6-Mut) at the doses used.

### EXAMPLE 2

This example provides possible toxicological studies and pharmacokinetic studies for the compounds of the present disclosure.

### Toxicological Studies

Establishing a No Observed Adverse Effect Level (NOAEL) of ExoBlock in mice to guide non-human primate studies to complete two species toxicity studies for further development and first-in-human dosing can be carried out. Dose-response relationships with various immune, renal, hepatic, and injection site toxicity endpoints can be evaluated in short-term, repeat-dose studies (28 daily sc doses in mice). Five dose levels can be evaluated in mice. Because immunotoxicity is critical part of immunotherapy, the potential of ExoBlock to cause such toxicity can be evaluated using both functional and non-functional endpoints. The possibility of renal and hepatic toxicity can be evaluated. The possible development of injection site toxicities resulting from the presence of high local accumulation can be evaluated.

Methods and Design: CD1 (ICR) mice can be used in this study. This outbred strain is a well-accepted animal model for general toxicology and immunotoxicology evaluations. Mice will be obtained from Charles River Laboratories (Portage, Mich.) at 4-5 weeks of age, and allowed to acclimate for 1 week prior to the study. Three mice can be housed per cage, on a 12 h light/dark cycle, at a temperature of 22±2° C. and humidity of 55±10%. Standard food and tap water will be provided ad libitum. Dose-response relationships with various immune, renal, hepatic, and injection site toxicity endpoints will be evaluated in short-term, repeat-dose studies. Dose selection can be guided by the anticipated clinical dose from efficacy studies. Five dose levels can be evaluated in mice and these ExoBlock doses include 2.56 mg/kg, 6.4 mg/kg, 25.6 mg/kg, 64 mg/kg and 256 mg/kg given sc. An appropriate dose can be evaluated in macaques to complete two species evaluation for further development (to be performed using matching funds). The overall study design and treatments groups for mice and primate are summarized in Table 2. Mice can receive daily doses of the assigned treatment for 28 consecutive days via sc injections (21 doses via sc daily for primates). The health status of all study animals can be monitored and documented on a daily basis via physical exams. Factors to be monitored include, but not limited to: body weight and presence of injection site reactions.

TABLE 2

| Organ/System | Toxicity Endpoints | Study Species |
|---|---|---|
| Immune system | TDAR Study: Anti-KLH IgM and | Mice, primates |
| | IgG titers | |
| | Peripheral blood cell counts | Mice, primates |
| | Lymphocyte immunophenotyping | Mice, primates |
| | Lymphoid organ structure: macro- | Mice |
| | and microscopic evaluation | |
| | Anti-lipid antibodies | Mice, primates |
| Liver | Liver structure: macro- and | Mice |
| | microscopic evaluation | |
| | Liver function | Mice, primates |
| Kidney | Kidney structure: macro- and | Mice |
| | microscopic evaluation | |
| | Kidney function | Mice, primates |
| Injection site | Injection site: physical and | Mice, primates |
| reactions | microscopic evaluation | (physical only) |
| | Plasma CK | Mice, primates |

Sample collection and handling: Non-terminal plasma and whole blood samples from mice can be collected via puncture of the saphenous vein into heparin or EDTA coated capillary tubes. Terminal plasma samples from mice can be collected by cardiac puncture into acid citrate dextrose (ACD: 85 mM sodium citrate, 110 mM D-glucose, 71 mM citric acid) at a 1:7 volume ratio. Serum samples can be collected by allowing whole blood with no anticoagulant to clot for 30 minutes at room temperature prior to centrifugation. EDTA- or citrate anti-coagulated plasma samples and serum samples will be collected similarly from rhesus macaques. All samples can either be analyzed immediately or stored at −80 ° C. until analysis. Immediately after exsanguination, mouse spleen, liver, kidney, and injection site skin samples will be harvested, weighed, and examined macroscopically. Tissue specimens can be fixed in 10% buffered formalin phosphate. Paraffin embedded sections (n=3/tissue/treatment group) can be stained with a Hematoxylin and Eosin (H&E) stain for histological examination. Histological specimen can be scored by an investigator blinded to the dosage information. Tissue sections can be evaluated for the following histopathological features of tissue injury: (a) inflammation, (b) fibrosis, and (c) cytopathic changes including the features of necrosis, apoptosis, cytoplasmic vacuolar change, hyperplasia, hypertrophy, atrophy, metaplasia, cell swelling, proteinaceous accumulations, fatty change and calcification. All of these features can be semi-quantitatively evaluated by a single reviewer according to the following scoring system: 0=absent; 1+=<5% of target; 2+=6-25% of target; 3+=>26% of target. Cell counts in murine peripheral blood can be analyzed using BC-2800 (Mindray, Mahwah, N.J.) and Sysmex XT2000iV (Sysmex, Lincolnshire, Ill.) auto hematology analyzers respectively. Serum chemistry markers can be used to evaluate functional health of the liver and kidneys. Mouse serum samples can be analyzed using a Vetscan VS2 (Abaxis diagnostics, Union city Calif.) or an Olympus AU400 (Beckman-Coulter, Brea, Calif.) analyzer. Plasma creatine kinase (CK) concentrations can be analyzed using a CK detection reagent. Functional T-cell dependent antibody response (TDAR) assay can be performed as described previously.

Statistical Analysis: Mean anti-KLH titer levels in mice can be compared using a one way ANOVA with Dunnett's post hoc analysis. Baseline and Day 18 or Day 22 mean anti-KLH titer levels in monkeys can be compared using a paired two sample t-test. Immunophenotyping data from mice can be compared using one way ANOVA with Dunnett's post hoc analysis. Mean plasma CK concentrations in ExoBlock-treated mice can be compared using a one-way ANOVA with Dunnett's post hoc analysis and a repeated measures ANOVA. p-values of less than 0.05 can be considered statistically significant.

### Pharmacokinetics Studies

Methods: The pharmacokinetics (PK) or time-course of plasma ExoBlock concentrations can be measured in NSG mice after i.v. or i.p. injection in short-term, repeat-dose studies. Five doses, ranging around the clinically relevant dose, can be evaluated in mice (e.g., 2.56, 6.4, 25.6, 64.6, and 245 mg/kg). A pilot study can be conducted with initial doses starting at 5, 10, and 50 mg/kg based on toxicity studies. The final 5 targeted dose levels may be modified to achieve a specific therapeutic effect or avoid toxicities. The wide range of dose levels can provide sufficient data to determine whether the PK is linear (i.e., net exposure is directly proportional to dose) or subject to capacity-limitation (i.e., nonlinear). A fixed volume of the drug in 100 μL can be injected i.v. or i.p., and average mg/kg/day dose can be calculated based on mean weight. NSG mice, both naïve (no tumor) and mice bearing DM6Mut tumor xenografts, can be administered daily doses of the assigned treatment for 28 consecutive days via i.p. injections. Non-terminal plasma and whole blood samples from mice can be collected, via vena puncture of the saphenous vein, into heparin or EDTA coated capillary tubes. Terminal plasma samples from mice can be collected by cardiac puncture into acid citrate dextrose (ACD: 85 mM sodium citrate, 110 mM D-glucose, 71 mM citric acid) at a 1:7 volume ratio. All samples can be either analyzed immediately or stored at −80 ° C. until analysis. These studies can be done by a clinical laboratory. The concentration of the drug in rodent plasma can be determined using a validated enzyme-linked immunosorbent assay (ELISA) assay.

Data Analysis: Measured plasma ExoBlock concentrations can be analyzed first using non-compartmental data analysis to calculate apparent PK parameters with the R statistical software package (https://www.r-project.org/). Area/moment analysis of drug concentrations following i.v. administration can be used to calculate the area under the plasma concentration-time curve (AUC), area under the first moment curve (AUMC), total systemic clearance (CL=Dose/AUC), steady-state volume of distribution (V_{SS}=CL·AUMC/AUC), and plasma half-life (T_{1/2}=0.693·AUMC/AUC). Drug bioavailability (F) after i.p. administration can be calculated as the ratio of respective AUC values (F=AUC_{i.p.}/AUC_{i.v.}). In order to describe the time-course of drug exposure, a minimal physiologically-based PK (mPBPK) model can be fitted to the measured plasma drug concentrations following both routes of administration. The base structural model can be slightly modified to include a first-order absorption process following i.p. drug administration. The mPBPK structure is constrained by physiological volumes and blood flows, which allows for the estimation of physiologically meaningful PK parameter values and forms a natural basis for scaling the model to predict drug exposures in humans. The PK/PD systems modeling software ADAPT Version 5 (BMSR, USC, Los Angeles, Calif.) can be used to develop the PK model. The PK data can be analyzed using a pooled approach with the maximum likelihood (ML) algorithm.

### EXAMPLE 3

This example provides possible dose, schedule, and delivery of compounds of the present disclosure.

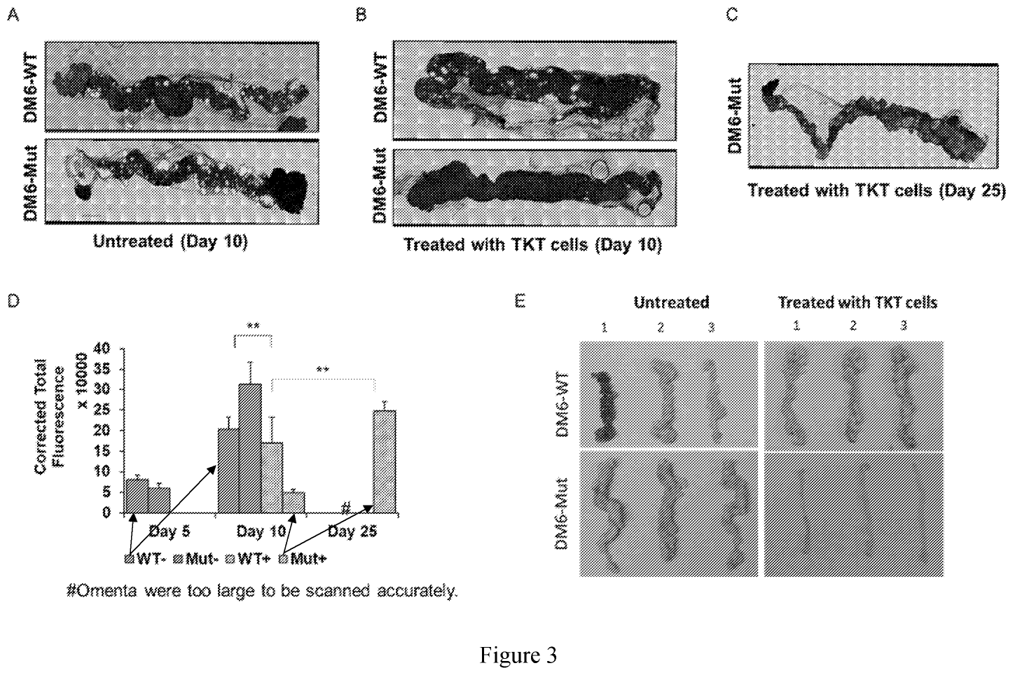

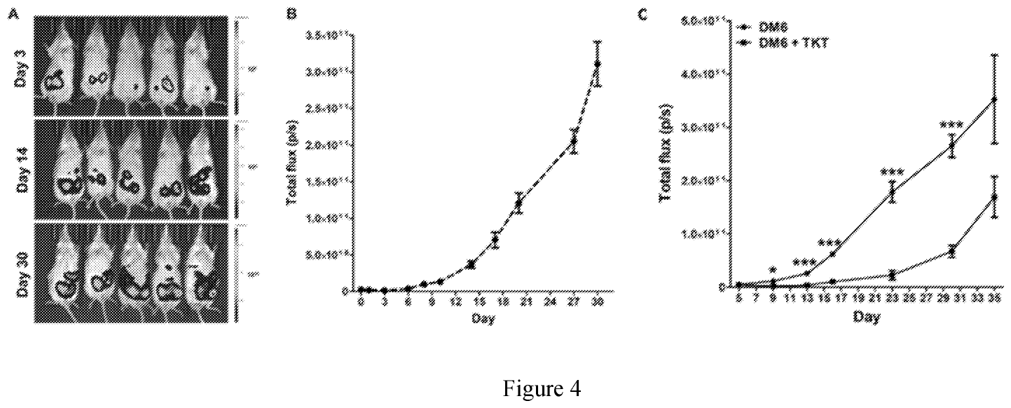

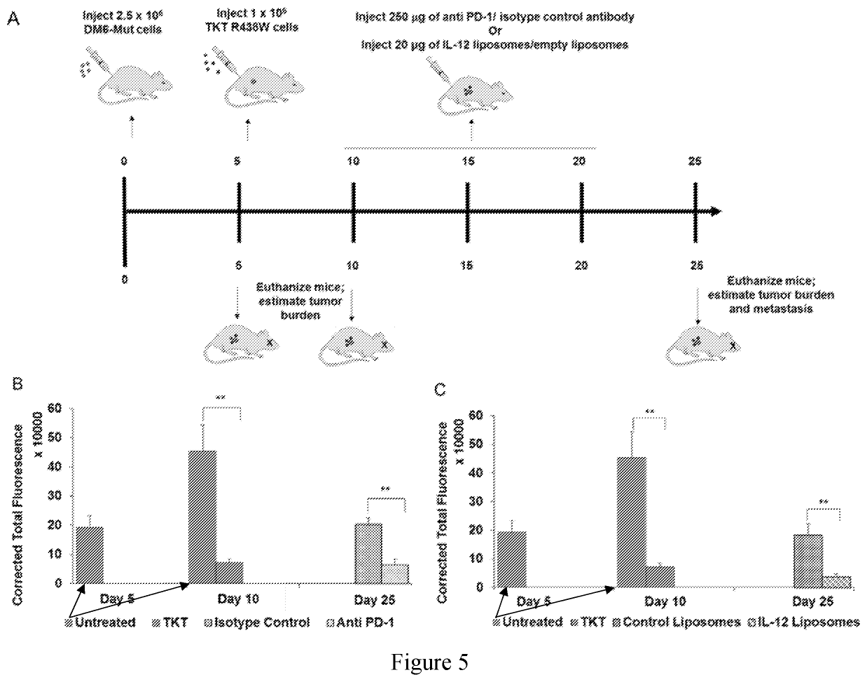

Rationale and Design: Using the X mouse model discussed above, it may be able to quantify changes in tumor burden (which directly reflect tumor-specific T cell function), that are associated with changes in drug doses, schedule and method of drug delivery, using both post-mortem GFP fluorescence imaging and live imaging of luciferase-dependent bioluminescence, Tumor burden can be determined every other day (following the adoptive transfer of T cells+/−drug) non-invasively in mice using bioluminescence of Luc+ DM6-Mut cells. With post-mortem imaging, tumor burden can be monitored at fixed time points i.e., days 5, 10, and 25. For these experiments, the optimal number of tumor cells that are injected i.p. into each mouse on day 0 (2.5×10⁶) and the number of tumor specific T cells that are injected on day 5 (0.5×10⁶) to achieve reproducible and statistically significant tumor suppression on day 10 resulting from the adoptive transfer of T cells has already been titrated and determined. By day 25, tumors escape this initial T cell suppression without further treatment. In the first schedule, mice will be treated with drug given i.p. on days 10, 15 and 20. It will begin with doses of 2.56 mg/kg, 6.4 mg/kg, 25.6 mg/kg, 64 mg/kg and 256 mg/kg. In the initial ExoBlock toxicity tests, NOAEL was observed at the 64 mg/kg drug dose. However, these doses may be adjusted depending on the more complete toxicity and PK studies described above. Anticipated decrease in tumor burden (in Luc+ DM6-Mut tumors) that are associated with increasing drug doses can be determined by live imaging every other day for 30 days. At intervals, mice can be injected with luciferin and the bioluminescence is quantified at an imaging facility. Data can be reported for each cohort as the arithmetic mean, SEM and p values as previously indicated above and in FIG. 3. The changes in tumor volume associated with drug treatment are also monitored on day 10 and day 25 using post-mortem imaging as outlined above and in FIG. 8.

### Methods

Set up of X mouse model: Globally immune deficient NSG mice will be used. Cohorts of 5 mice (treated and untreated) can be injected i.p. with the GFP+ Luc+ DM6-Mut tumor cells on day 0. Five days after tumor xenografts are generated in the greater omentum, mice can be injected with the tumor specific T cells (TKT R438W). TKT cells cannot be given to the control group. Treatment of experimental mice can begin on day 10 with different schedules, doses and delivery methods. Live imaging of the mice can begin on day 1 and can be continued every other day for 25 days. Post mortem imaging can be done on days 5, 10, and 25. Cohorts of mice at these time points can be euthanized and the greater omenta can be removed to prepare whole mounts in PBS. These can then be scanned for GFP fluorescence using a Leica DM 6B upright fluorescence microscope. The fluorescence can then be quantified using ImageJ software. Corrected total fluorescence data (after subtracting the background for each omentum) is plotted and statistically analyzed as shown in FIG. 8 at the time points indicated in the design above.

ExoBlock dose escalation studies: Control cohorts of mice include mice given (a) tumors but not TKT cells (b) tumors and TKT cells but no drug, and (c) the tumor and the highest dose of ExoBlock (64 mg/kg). The experimental cohorts can be given tumors, TKT cells and increasing doses of drug can be monitored and compared for changes in tumor burdens. Treatment of mice with the drug can begin on day 10 and can be repeated on days 15 and 20. This schedule can be adjusted in the subsequent schedule change experiments. The drug doses may change depending upon the toxicity and PK studies described herein.

Treatment scheduling: ExoBlock can be injected every 5 days for the initial experiments, and the frequency of injections can be modified depending upon data available from the PK studies, including half-life of ExoBlock in the mouse. For an initial experiment, 3 different schedules can be tested, which include starting the injection of ExoBlock either before (days 3, 8, 13, and 18), simultaneously (days 5, 10, 15, and 20), or after (days 10, 15, and 20 as was used previously) the injection of T cells.

Design and use of PK/PD model to predict the optimal dose and schedule for ExoBlock to reduce tumor burdens in X mouse model: a PK/PD model was designed. This model is specifically designed to link drug concentration profiles to the time-course of tumor growth kinetics and will be used to predict an optimal dose and schedule for ExoBlock to most effectively enhance the anti-tumor activity of tumor specific T cells resulting in the suppression of tumor in the primary site (omentum) and in preventing the dissemination of the tumor to other organ sites. In this model, the data obtained in the X mouse model studies (using live imaging and monitoring changes in tumor burdens every other day for 30 days) are used to do generate an exposure-response relationship of ExoBlock with enhanced tumor suppression mediated by the tumor specific T cells at different drug doses and treatment schedules. The PK model and estimated parameters developed can be fixed to serve as a driving function in the PD model that links ExoBlock concentrations to enhanced therapeutic efficacies. A hierarchical series of PD models will be applied to determine the best structure for coupling the PK and PD tumor response data. Parameters can be estimated in ADAPT5 and include rate constants (with or without capacity limits) associated with unperturbed tumor growth kinetics and effect parameters, such as a second-order T cell mediated tumor suppression rate constant and an interaction parameter quantifying the ExoBlock cell interaction. The final model can be validated by comparing simulated enhanced tumor suppression curves with observed suppression profiles. The predicted optimal treatment regime can be tested in both the live and post-mortem imaging protocols.

Validation of tumor suppression that is determined by quantification of fluorescence and bioluminescence by histopathology and immunochemistry. Mice can be sacrificed at selected intervals and omenta can be removed, fixed and stained, and slides examined histologically for the evidence of tumors. These tissue sections will be stained with a melanoma specific Mel A antibody to estimate and confirm large changes in tumor amounts predicted with fluorescence and bioluminescence.

It has been determined that ExoBlock has no direct suppressive effect on DM6-Mut cells in vitro. An additional control group has been included in the methodology (tumor cells+ExoBlock at the highest dose used i.e., 64 mg/kg) to account for any direct effects of drug on tumor. There is evidence that exosomes expressing the ExoBlock targeted marker, PS, are released from the DM6-Mut tumors in the X mouse model and that these exosomes are immune suppressive. By using a nanoparticle tracking analysis (NTA) tool (the ZetaView) with a laser, it will be able to quantify the number of PS+ exosomes. It can be possible now to establish that there is a loss or decrease in immune suppressive properties of equimolar amounts of exosomes isolated from the xenografts with or without ExoBlock treatment.

7 different tumor specific T cells derived from 3 different melanoma patients that recognize and specifically kill tumor target cells expressing the cognate tumor peptide are available. In addition, there are T cells specific for G280-9V, a peptide derived from the gp100 protein that is universally present on the surface of primary patient-derived melanomas as well on DM6-Mut cells. ExoBlock can be tested in these systems to confirm its universal applicability. These additional tumor-specific T cells can be used in place of the TKT cells.

To improve the therapeutic efficacy of a checkpoint blocking antibody (e.g., nivolumab) it can be combined the ExoBlock regimen developed above.

### EXAMPLE 4

This example provides possible examples of using the compounds of the present disclosure.

Rationale and Design: The blockade of PD-1 can induce sustained clinical responses in some cancer patients, but how they function in vivo and why they fail to produce any response or durable responses in many patients remain incompletely understood. The tumor microenvironment is complex and includes many immune suppressive cells and molecules that can co-op T cell function. One of these immune suppressive factors is the immune suppressive exosomes that has been determined to act similarly to other checkpoint molecules. Metastatic melanomas in cancer patients release exosomes that express PD-L1 on their surface, suppress the function of CD8 T cells and facilitate tumor growth. Multiple different exosomes present in tumor microenvironments may contribute to the failure of checkpoint therapies and that a blockade of multiple subsets of immune suppressive exosomes could enhance the efficacy of checkpoint blocking therapies and improve clinical response rates and the durability of these responses. It has already been established with the X mouse model that treatment of mice with anti-PD-1 antibody (nivolumab) enhanced the tumor suppression and delayed, but did not prevent tumor recurrence. The combination of the exosome blocking drug with anti-PD-1 may enhance the efficacy of the checkpoint blocking therapy.

### Methods

The steps outlined above for the X mouse set up to monitor the effects of treatment with ExoBlock can be essentially the same that is used here to quantify the ability of anti-PD-1 to inhibit tumor progression and compare this to the ability of the combination of ExoBlock and anti-PD-1 to inhibit tumor growth.

Cohorts of 5 mice bearing tumors that have received T cells on day 5 can be treated with (a) nivolumab 10 mg/kg on days 10, 15, and 20, (b) with the same dose of an isotype control at the same regimen, (c) a combination of nivolumab 10 mg/kg on days 10, 15, and 20 with ExoBlock at a dose, delivery method and schedule that was identified as optimal, (d) ExoBlock at the optimal treatment regimen only, and a cohort of control mice that are injected on day 5 with tumor but receive no treatment. An additional endpoint used here can be survival (or day of euthanasia). The mice used in live imaging studies may not be euthanized on day 30, and can be monitored until they develop humane endpoints i.e., clinical signs of distress, neoplasia, or moribundity that necessitate euthanasia.

All mice can be monitored every other day for 25 days for changes in tumor burden by live imaging and determination of bioluminescence as indicated above. In separate experiments, the same groups are set up and tumor burdens can be quantified at days 5, 10, and 25 by measuring the GFP fluorescence.

The corrected total fluorescence can be calculated by subtracting the background for each omentum. Data can be plotted as Mean±SEM. Student's t test will be used to establish statistical significance. The percentage reduction in tumor burden (represented by the CTF) can be calculated for the single treatment (nivolumab or ExoBlock) and the combination cohorts (nivolumab+ExoBlock), compared to the cohort which receives only TKT cells. For the survival endpoint, the mean lifespan of each cohort can be calculated in addition to plotting a Kaplan-Meier curve. A significant (p<0.05) improvement in the reduction of tumor burden or increase in life span in the combination cohort can be interpreted as an additive effect.

### EXAMPLE 5

The following example provides a description of synthesis of compounds of the present disclosure.

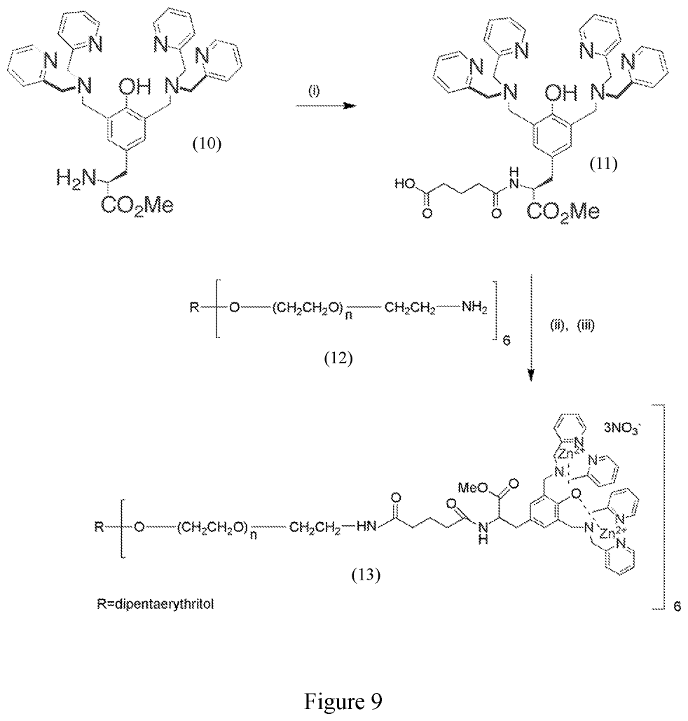

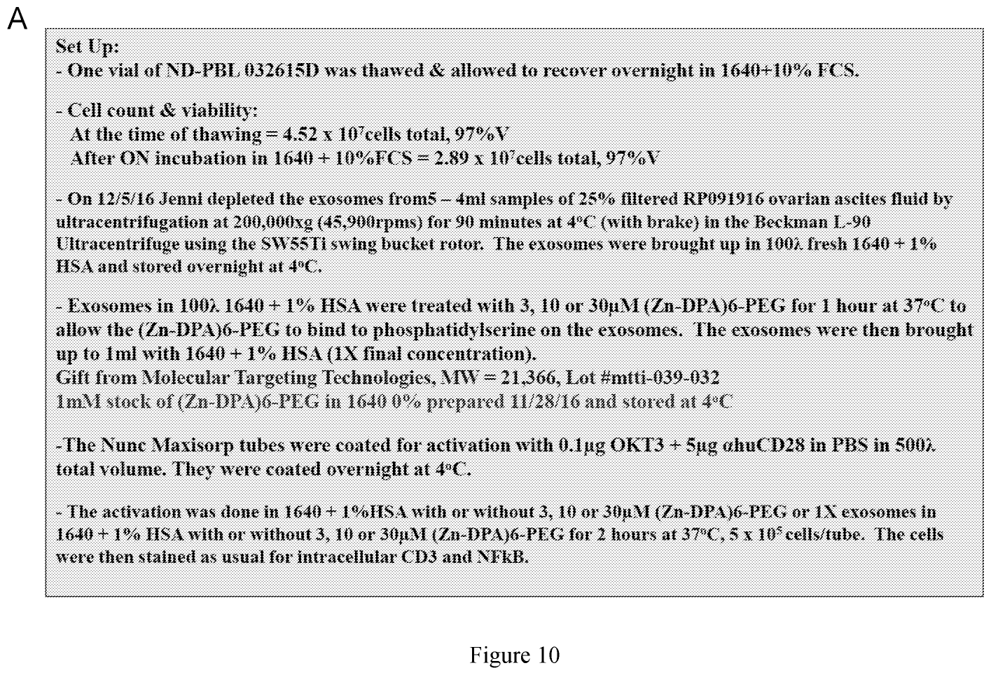

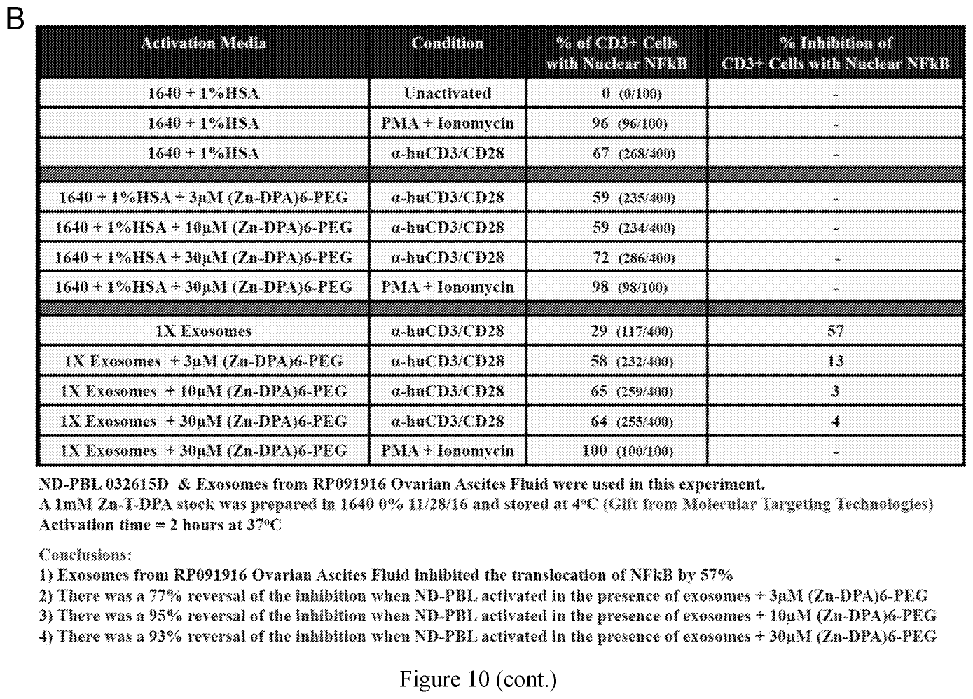

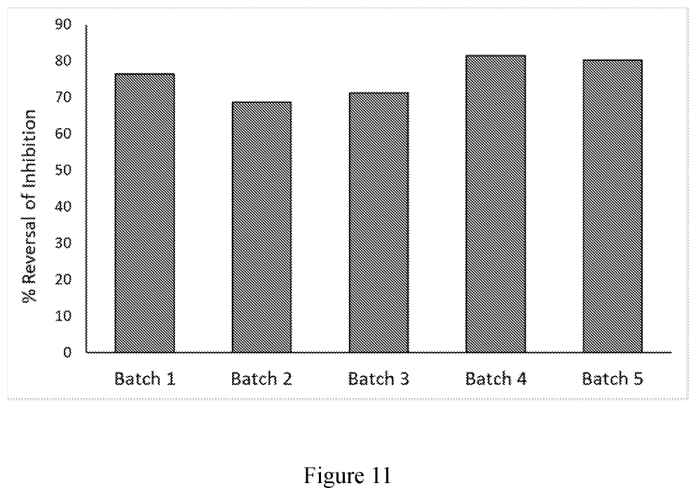

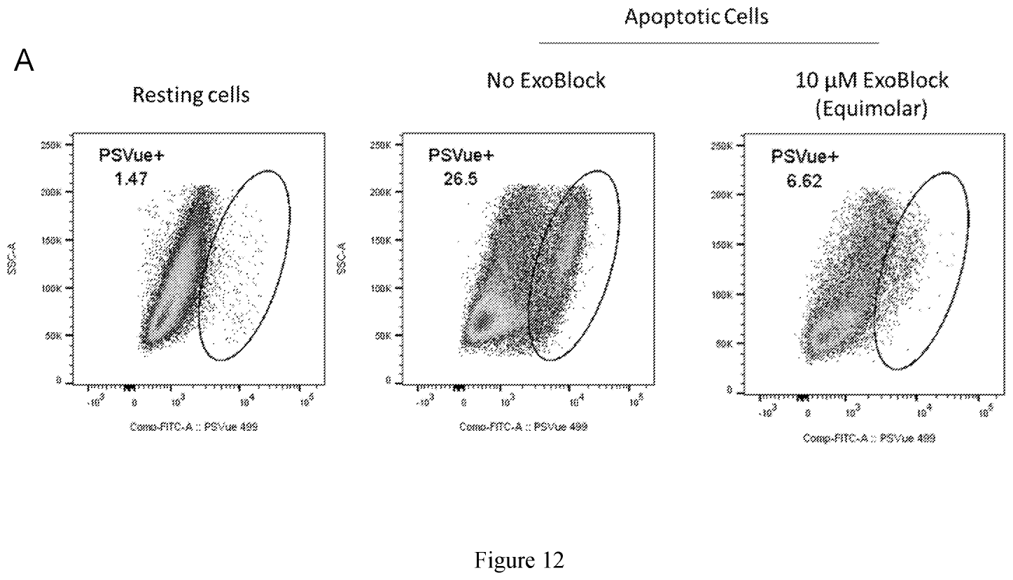

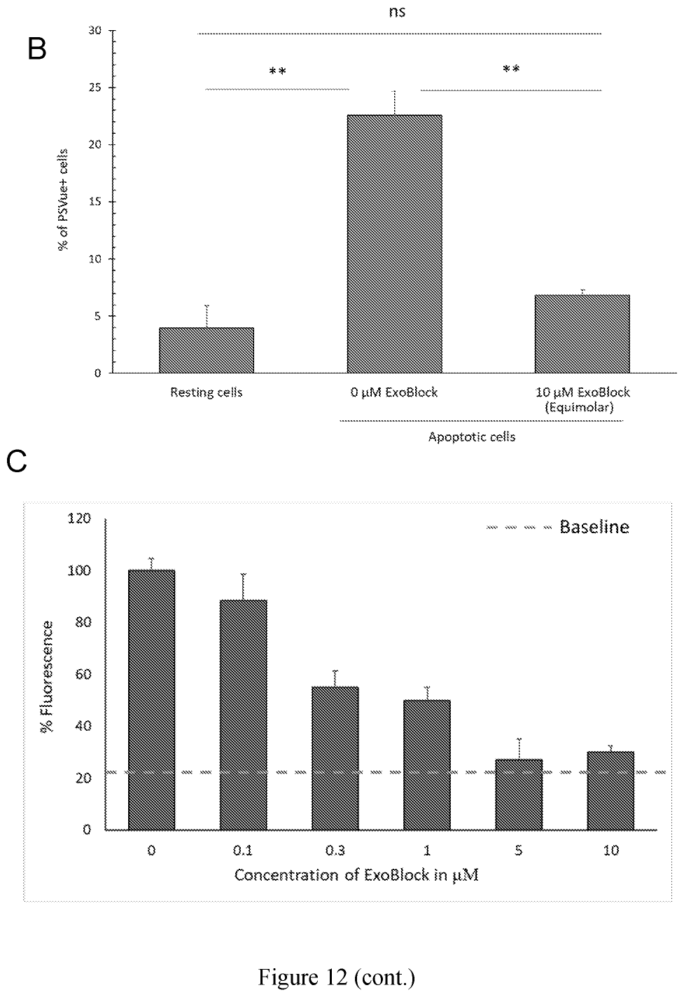

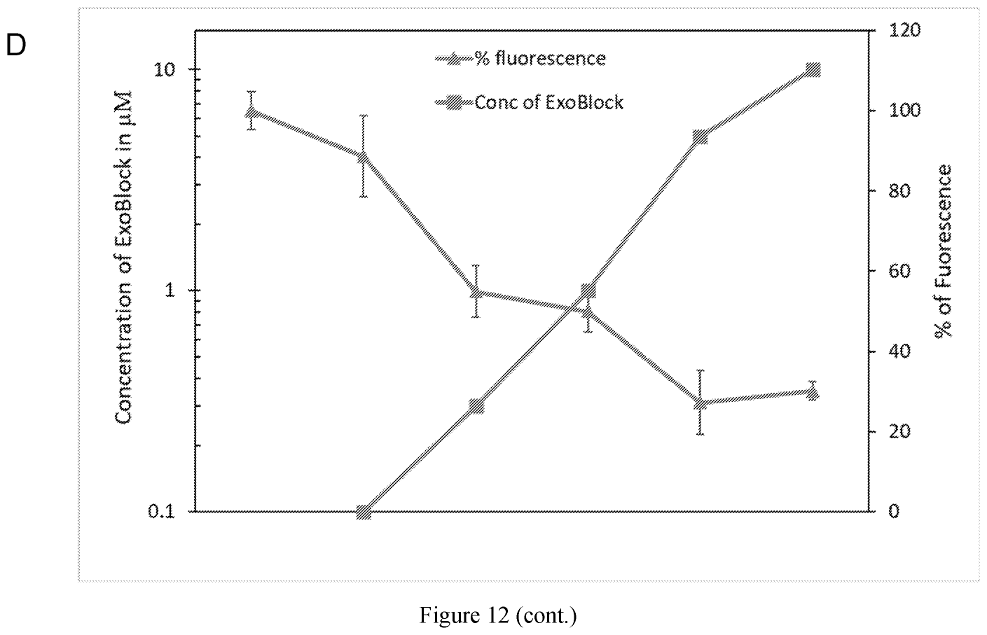

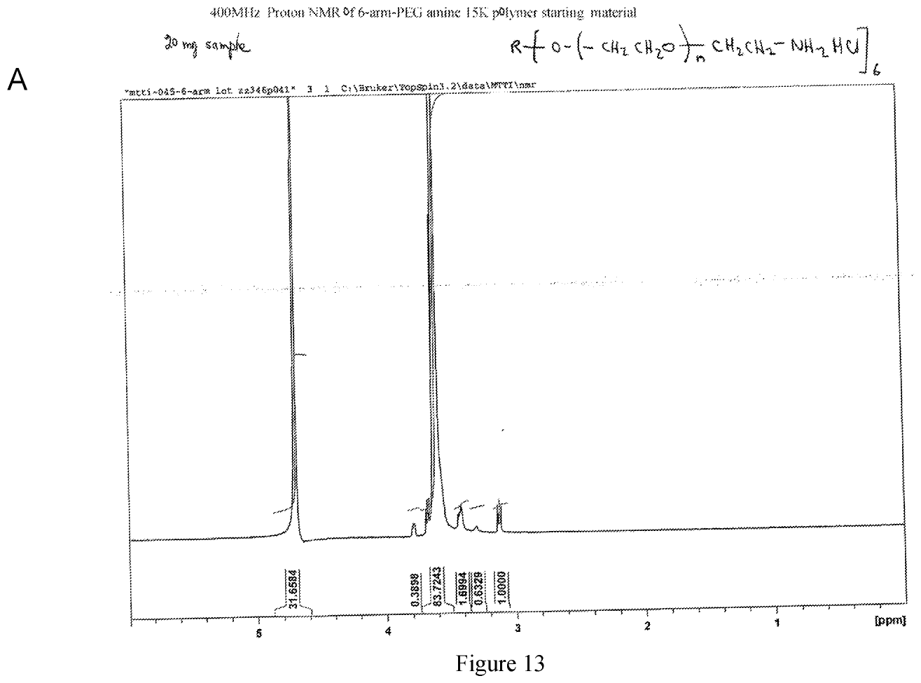

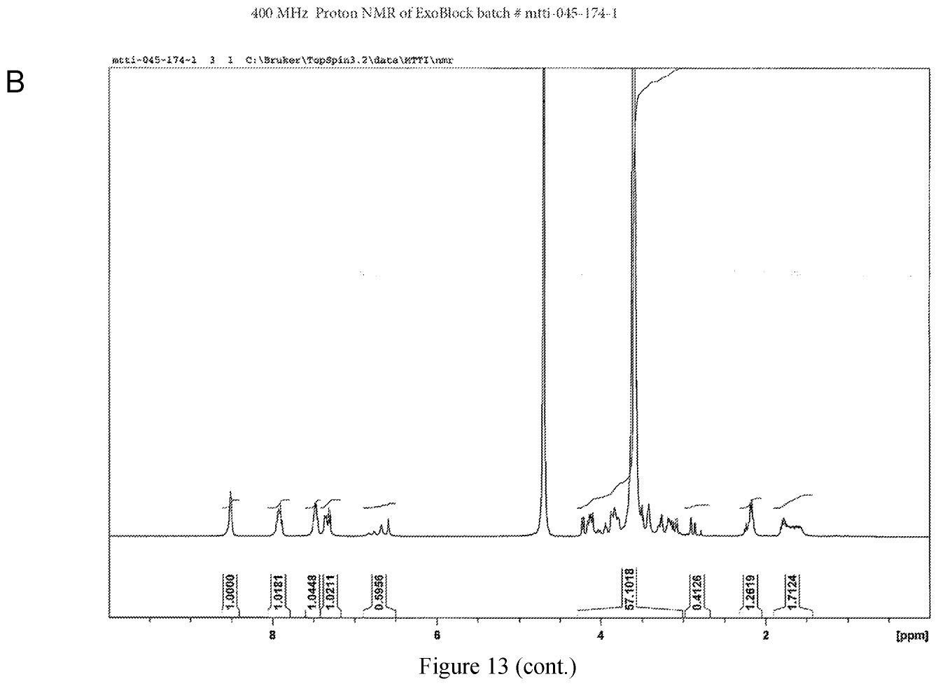

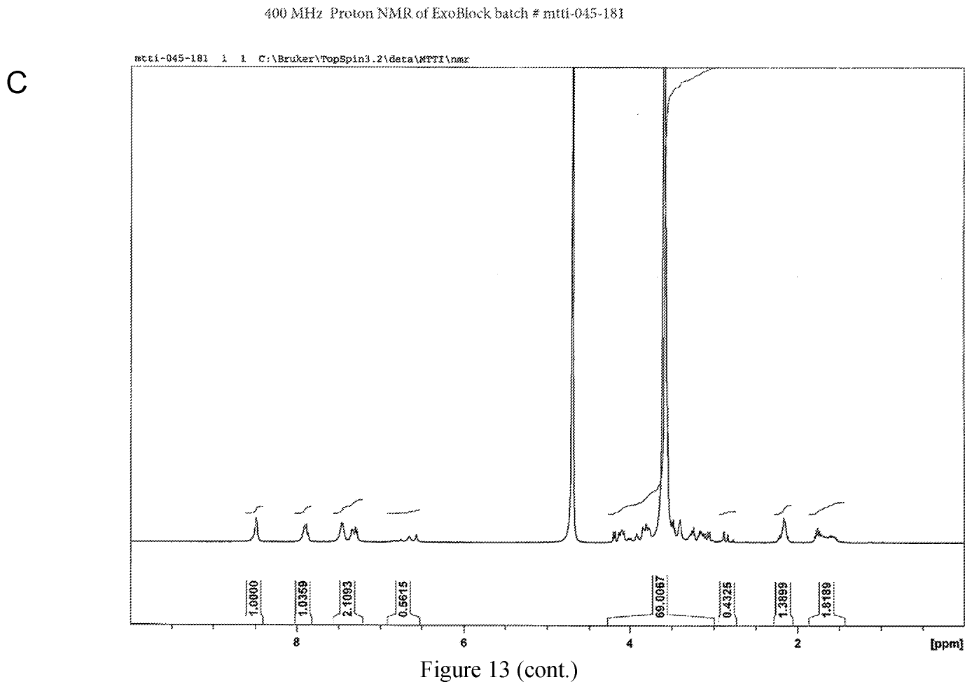

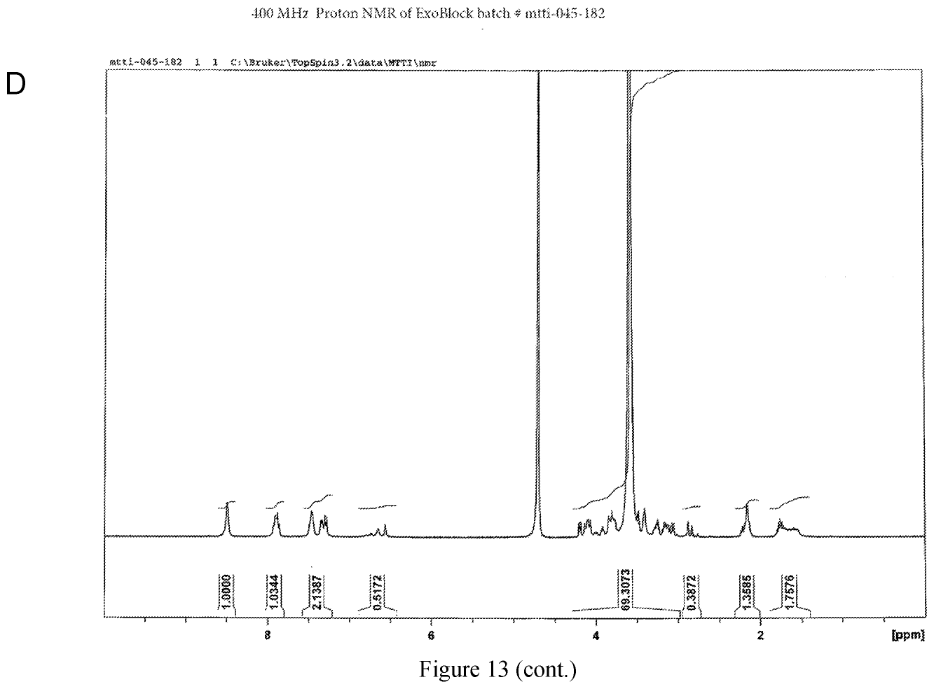

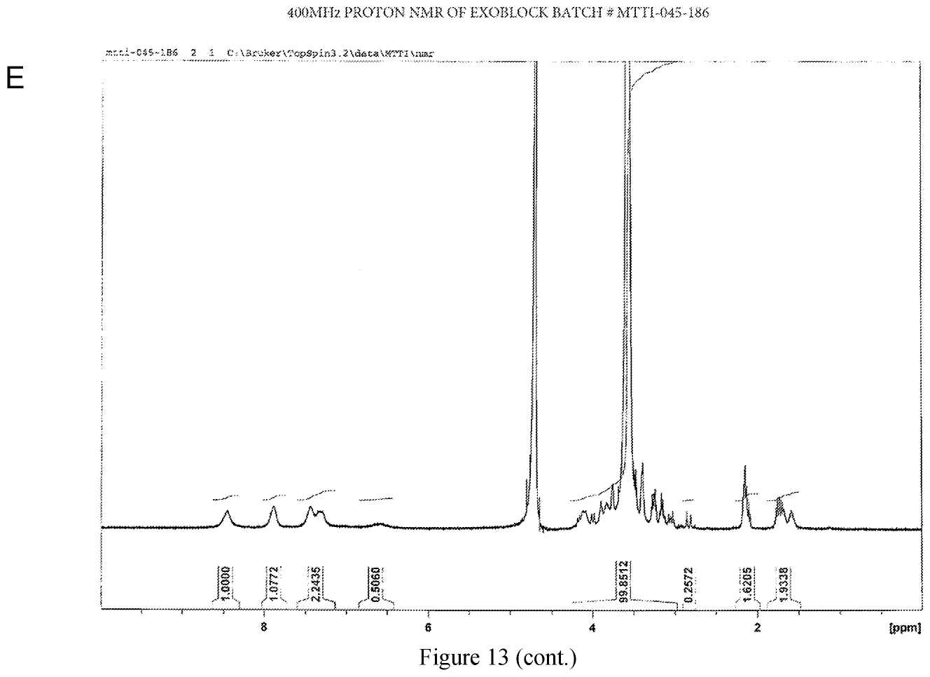

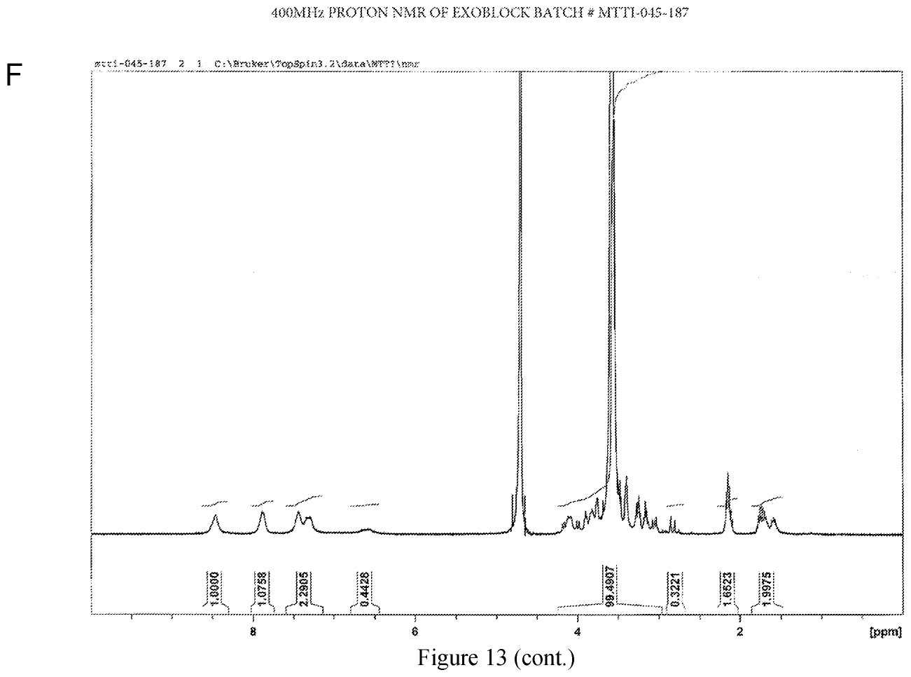

Preparation of 6-arm Zn-DPA-DP-15K. 2,2′-Dipicolylamine (DPA) is prepared in 5 synthetic steps and reacted with glutaric anhydride to provide DPA-acid. Activation of DPA-acid with sulfo-N-hyroxysuccinimide and 1-ethyl-3-(3-dimethyiaminopropylcarbodiimide (EDC) forms the activated ester in situ which is then treated with 6-ARM(DP)-NH2-15K and finally with zinc nitrate hexahydrate to furnish Zn-DPA-DP-15K. See FIG. 9.

Preparation of 6-arm Zn-T-DPA-DP-15K. Tyrosine-DPA is prepared in 2 steps and reacted with glutaric anhydride to provide T-DPA-acid. Activation of T-DPA-acid with sulfo-N-hyroxysuccinimide and 1-ethyl-3-(3-dimethyiaminopropylcarbodiimide (EDC) forms the activated ester in situ which is then treated with 6-ARM(DP)-NH2-15K and finally with zinc nitrate hexahydrate to furnish Zn-T-DPA-DP-15K.

### Detailed Experimental Procedure

DPA (0.523 g, 0.891 mmol) and glutaric anhydride (0.107 g, 0.935 mmol) are stirred in 20 mL of anhydrous chloroform overnight. The solvent is removed by rotary evaporation and the resultant oil (0.593 g) characterized by proton NMR. 0.593 g of is stirred with S-NHS (0.234 g, 1.078 mmol) and EDC (0.189 g, 0.984 mmol) in DMF (12 mL) overnight. 6-ARM(DP)-NH2-15K (0.45 g, 29.7 μmol, Jenkem Technology) in DMF (10 mL) containing N,N-diisopropylethylamine (50 μL) is then added and the mixture stirred at room temperature overnight. The solvent is then removed by rotary evaporation and the residue taken up in 40 mL of methanol containing zinc nitrate hexahydrate (0.630 g, 2.12 mmol) and stirred overnight. The solvent is then removed and the residue taken up in 30 mL of water and placed in dialyzer bags of molecular weight cut-off=8-10K and dialyzed against 3 L of water with 3 changes of water. The solution is then filtered through a 0.2 μm filter and freeze-dried on a lyophilizer overnight to provide 0.56 g of as a white solid.

TABLE 3
Methods for the Characterization of ExoBlock

| Parameter | Methodology and Specifications |
|---|---|
| Appearance: Color & Form | White powder |
| Molecular Weight | By gel permeation chromatography |
| | (GPC) with detection at |
| | 210 nm and 260 nm; and by MALDI-TOF |
| | mass spectrometry. Samples will be sent to |
| | AA labs, LLC, San Diego, CA |
| Structure Characterization | By ¹H and ¹³C NMR. Spectrum should |
| | be consistent with structure |
| Purity | By GPC and HPLC |
| Elemental Composition | CHN and Zn Content |
| Water Content | Karl-Fischer testing to determine water content |

### EXAMPLE 6

The following example provides characterization of compounds of the present disclosure.

Five batches of ExoBlock were analyzed using a standard colorimetric 2,4,6-trinitrobenzene sulphonic acid (TNBS) assay which uses absorbance at 340 nm to detect for free amino groups present and compared against a standard curve generated from a series known concentrations of the 6-arm-PEG amino starting polymer (MW=15K).

The assay yielded the following results:
Batch 1 (lot #mtti-045-174-1): 1.0% free amino groups
Batch 2 (lot #mtti-045-181): 2.7% free amino groups
Batch 3 (lot #mtti-045-182): 2.2% free amino groups
Batch 4 (lot #mtti-045-186): 2.4% free amino groups
Batch 5 (lot #mtti-045-187): 2.4% free amino groups

These data show the product was produced with >97% of initially available amines on the 6-arm polymer reacted with ZnDPA moieties.

Although the present disclosure has been described with respect to one or more particular examples, it will be understood that other examples of the present disclosure may be made without departing from the scope of the present disclosure.

1. A compound having the following structure: 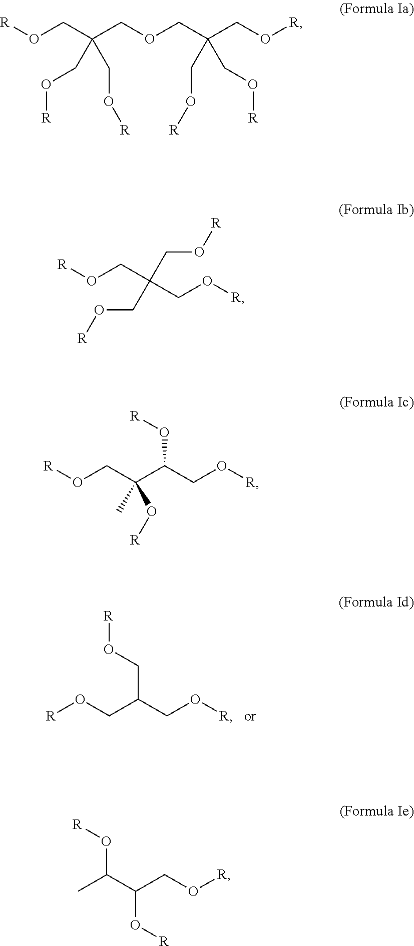
wherein each R is independently at each occurrence hydrogen or comprises a poly(ethylene glycol) (PEG) group or an ethylene glycol group, a linker group, and an end group.

2. The compound of claim 1, wherein the linker group has the following structure: 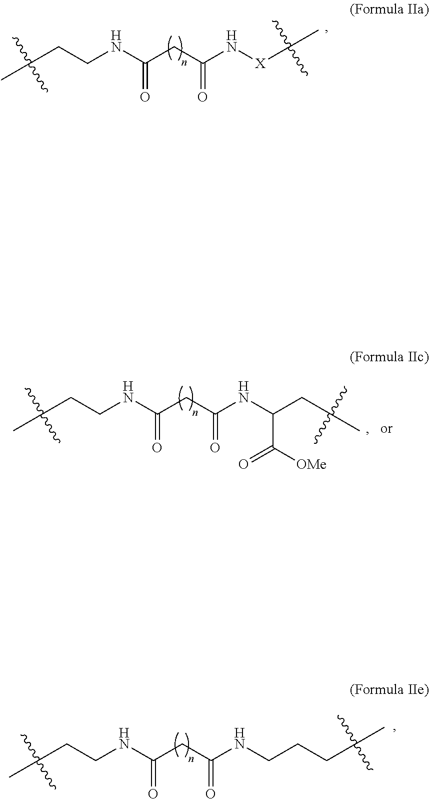
wherein X is a spacer group.

3. The compound of claim 1, wherein the end group has the following structure: 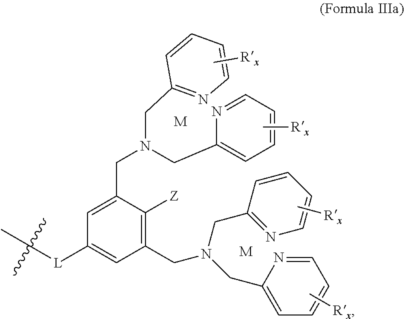
wherein L is O or —CH₂— and Z is OH, O, or H, wherein O is chelated to M, R′ is independently at each occurrence chosen from hydrogen, halogens, aliphatic groups, aryl groups, alkoxide groups, amine groups, carboxylate groups, carboxylic acids, ether groups, alcohol groups, alkyne groups, and combinations thereof, and x is 1, 2, 3, or 4.

4. The compound of claim 3, wherein the end group has the following structure:

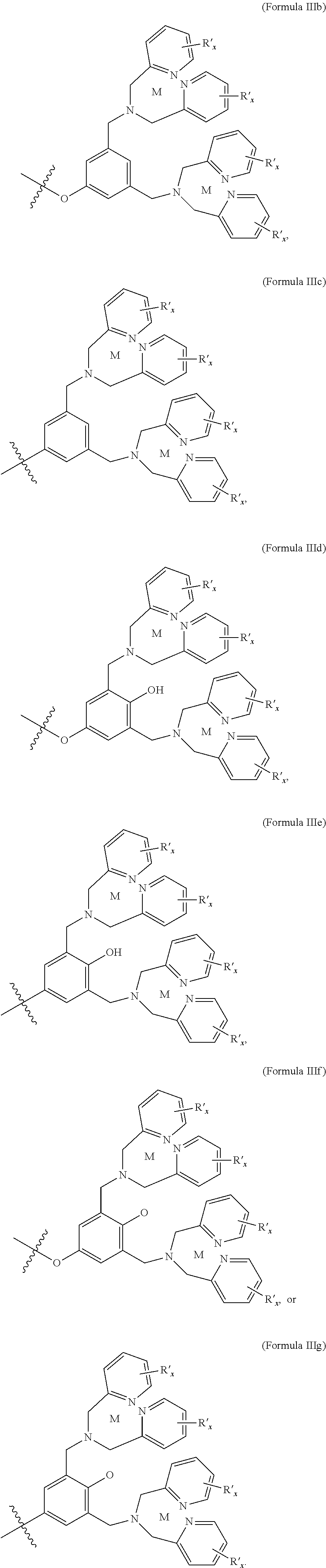

5. The compound of claim 3, wherein the end group has the following structure:

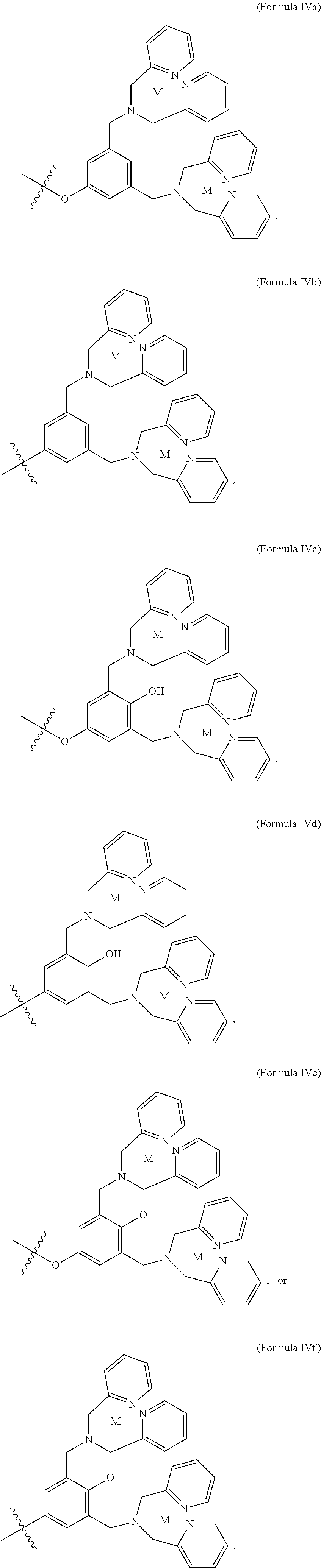

6. The compound of claim 1, wherein the compound has the following structure: 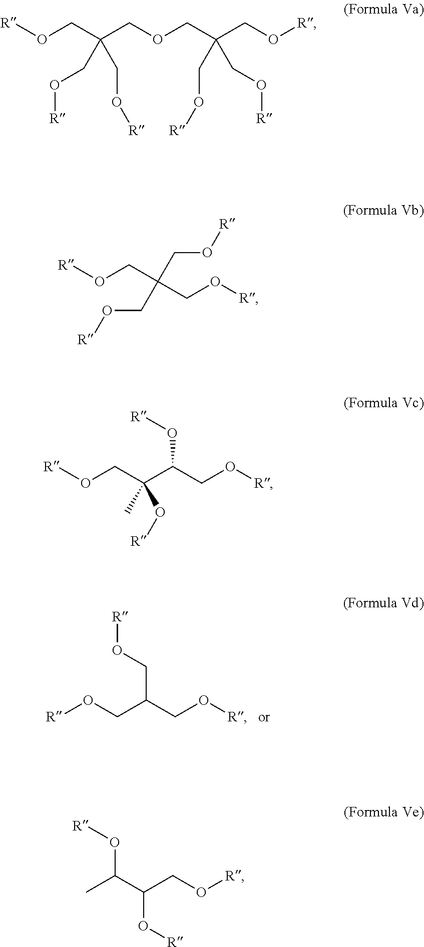
wherein R″ is independently at each occurrence H or 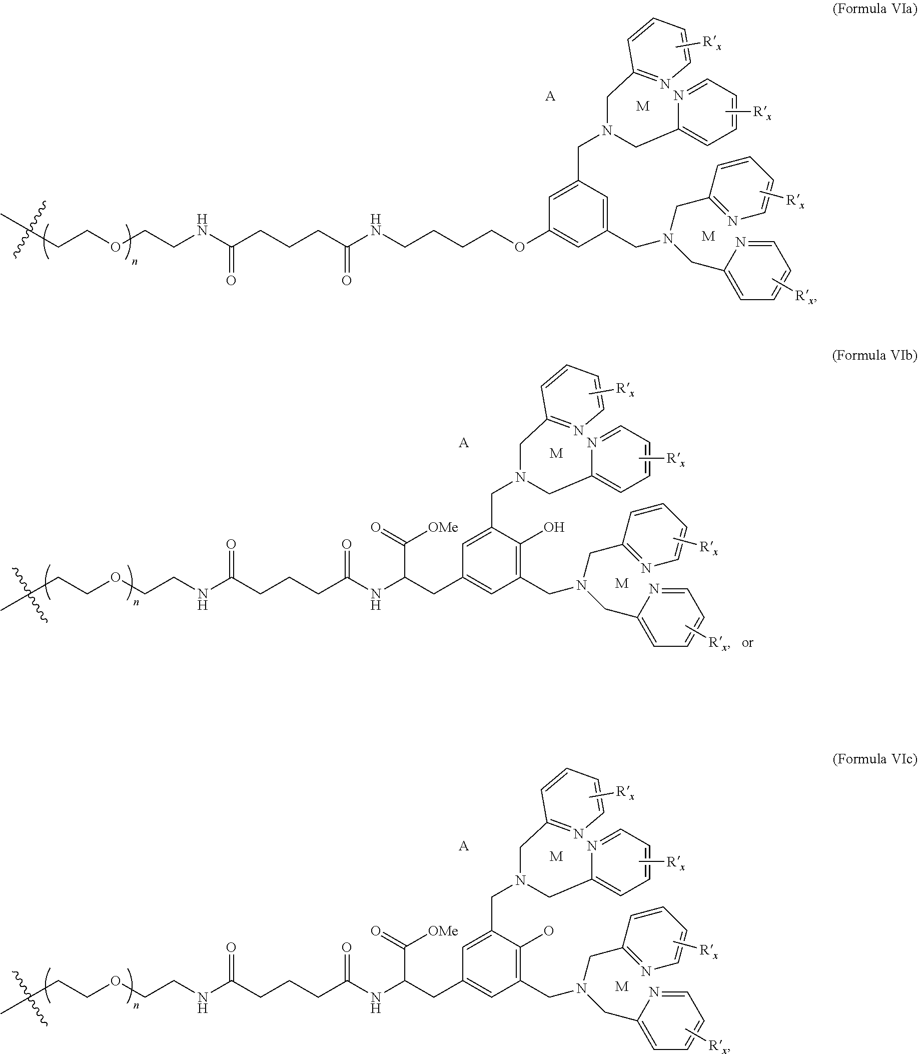
wherein M is a divalent cation, R′ is independently at each occurrence chosen from halogens, aliphatic groups, aryl groups, alkoxide groups, amine groups, carboxylate groups, carboxylic acids, ether groups, alcohol groups, alkyne groups, and combinations thereof, A is one or more counter anions, x is 1, 2, 3, or 4, and n is 1-500.

7. The compound of claim 6, wherein the compound has the following structure: 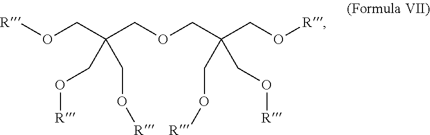
wherein R″′ is independently at each occurrence H or 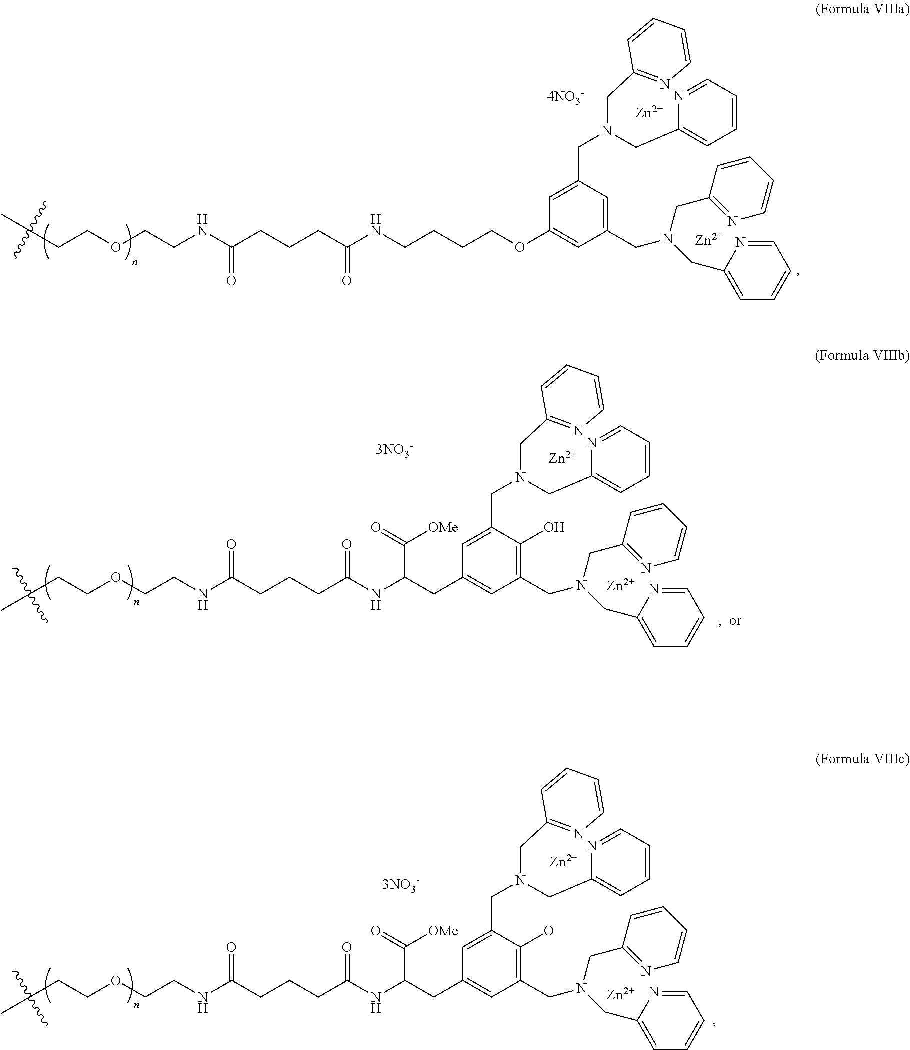
and n is 1-500.

8. A composition comprising a compound of claim 1 and one or more pharmaceutically acceptable carriers.

9. The composition of claim 8, further comprising an anti-PD1 antibody, an anti-CTLA-4 antibody, an anti-LAG3 antibody, an anti-TIM3 antibody, or a combination thereof.

10. The composition of claim 9, wherein the anti-PD1 antibody is chosen from nivolumab, pembrolizumab, durvalumab, camrelizumab, cemiplimab, sintilimab, toripalimab, and combinations thereof.

11. A liposome composition, wherein the liposomes have incorporated therein a compound claim 1.

12. The liposome composition of claim 11, wherein the liposome has a monolayer or bilayer and the monolayer or bilayer comprise phosphatidylcholine (“PC”) and/or phosphatidylglycerol (“PG”) and, optionally, cholesterol.

13. A method of treating an individual in need of treatment for cancer, comprising administering to the individual one or more compounds of claim 1 or one or more compositions comprising a compound of claim 1.

14. The method of claim 13, wherein the cancer is a solid tumor, leukemia, lymphoma, or a combination thereof.

15. The method of claim 14, wherein the solid tumor is associated with melanoma.

16. The method of claim 13, wherein the composition is a liposomal composition.

17. The method of claim 13, wherein the individual is a human.

18. The method of claim 13, wherein the individual is a non-human mammal.
